(19) United States
(12) Patent Application Publication
(10) Pub. No.: US 2015/0017092 A1
(43) Pub. Date: Jan. 15, 2015

(54) DEVICES AND METHODS FOR DETERMINING SENSITIVITY TO RADIATION

(71) Applicant: The Regents of the University of California, Oakland, CA (US)

(72) Inventors: Sylvain V. Costes, Albany, CA (US); Rafael Gomez-Sjoberg, Menlo Park, CA (US); Steven M. Yannone, Concord, CA (US)

(21) Appl. No.: 14/378,617

(22) PCT Filed: Mar. 14, 2013
(86) PCT No.: PCT/US2013/031727

§ 371 Date: Aug. 13, 2014

Related U.S. Application Data
(60) Provisional application No. 61/611,461, filed on Mar. 15, 2012.

(51) Int. Cl. G01N 33/50; A61N 5/10; A61B 5/00

(52) U.S. Cl. CPC G01N 33/5014; A61B 5/0033; A61N 5/1067
USPC 424/1.11; 435/287.2; 435/7.1; 600/407; 600/1

(57) ABSTRACT

Systems and methods for determining the sensitivity of cells (and/or a subject) to ionizing radiation are provided. The systems can comprise a microfluidic device comprising a plurality of microfluidic cavities each configured to contain cells; a source of ionizing radiation configured to deliver ionizing radiation to cells in the microfluidic cavities; and an imaging system configured to detect radiation-induced foci in cells when they are disposed in the microfluidic cavities. The methods can involve contacting a biological sample comprising cells from a subject with ionizing radiation; detecting and quantifying radiation induced foci in the cells at least two different time points; and determining a repair kinetic for radiation induced foci that is a measure of the rate of disappearance of the foci. Methodologies are also provided for in-home blood collection and fixation of nucleated blood cells in a manner to preserve health and fitness biomarkers inherent to these cells.

### CROSS-REFERENCE TO RELATED APPLICATIONS

This application claims priority to and benefit of U.S. Ser. No. 61/611,461, filed on Mar. 15, 2012, which is incorporated herein by reference in its entirety for all purposes.

### STATEMENT OF GOVERNMENTAL SUPPORT

This work was supported in part by Grant No DE-AC02-05CH11231 from the Department of Energy (DOE). The Government has certain rights in this invention.

### BACKGROUND

Discovering mechanisms involving radiation-induced cancer will help refine risk assessment of ionizing and/or non-ionizing radiation at very low dose levels, where epidemiological data cannot resolve risk uncertainties. Human variability is currently completely ignored in the management of health risk to ionizing radiation, using a “one fit all” risk model approach. However, it is quite common to observe severe skin reactions in patients treated for radiotherapy after a few sessions. In such cases, a course of action must be taken for this patient, when this could have been avoided if there was a screening method to predict how that patient would have reacted.

For less acute doses (e.g. medical imaging, airport scanning, and the like), long term effects such as cancer are more difficult to be directly linked to a specific exposure. Epidemiologic studies suggest that it takes about 20 years from a radiation-induced mutation to become a cancer. Therefore, cancer risk estimation continues to be an active field of research as it is very difficult to prove causality at the individual level. The classic assay for cancer risk from radiation has been to measure excess levels of chromosome rearrangement (by FISH or micronucleus assay) following ionizing radiation. There is a large body of literature suggesting cancer incidence in mice correlate well with high levels of chromosomal rearrangements measured days after an exposure to ionizing radiation. However, cytogenetic assays are slow, not sensitive (need high doses) and labor intensive, making them difficult to be used as a screening tool. Therefore, gene expression profiling would be the preferred method for screening as it can be automatized and used in a high throughput manner. However, gene expression can be quite misleading as their profiles do not always translate into an actual effect in the human body. For example, having a gene being highly expressed does not necessarily mean its corresponding protein will be transcribed, and often leads to false positive or false negative.

Finally, DNA repair deficiency is linked in general to increased cancer risk and thus determining this efficiency in a large population may on the long run be a great preventive tool (i.e. people at risk could take action such as dietary supplement such as anti-oxidants, or modified behavior such as avoiding long UV exposure, etc. . . . )

### SUMMARY

In various embodiments systems and/methods for determining the sensitivity of cells (and by implication the subject from whom the cells are derived) to ionizing radiation or to non-ionizing radiation are provided. In certain embodiments the systems comprise a microfluidics device comprising a plurality of microfluidic cavities each configured to contain cells; a source of ionizing radiation configured to deliver the ionizing radiation to cells in the microfluidic cavities; and an imaging system configured to detect radiation-induced foci in the cells when they are disposed in the microfluidic cavities. In certain embodiments the source of radiation is a source of ionizing radiation (e.g., a radionuclide or an x-ray source). In certain embodiments the source of ionizing radiation is a mini X-ray tube. In certain embodiments the source of radiation is a source of non-ionizing radiation (e.g., a UV source). In certain embodiments the microfluidic device comprises at least eight microcavity cells for each sensitivity determination that is to be performed. In certain embodiments the microfluidic device is configured to provide a plurality of sensitivity determinations. In certain embodiments the microfluidic device is configured to provide at least four different sensitivity determinations. In certain embodiments the at least eight microcavity cells for each sensitivity determination are disposed along a line on the microfluidic device. In certain embodiments the microfluidic device is operably coupled to or further comprises a cell separator. In certain embodiments the cell separator is configured to separate lymphocytes from a blood or blood fraction sample and deliver the lymphocytes into the microfluidic cavities. In certain embodiments the separator lyses erythrocytes and isolates leukocytes. In certain embodiments channels or chambers in the cell separator are coupled to the microcavities by microchannels and configured to deliver the lymphocytes from the separator into the microcavities. In certain embodiments the microfluidics device comprises a fabricated block within which are formed, embedded or molded, one or more fluid-tight channels. In certain embodiments the block material from which the device is fabricated is selected from the group consisting of polydimethylsiloxane (PDMS), polyolefin plastomer (POP), perfluoropolyethylene (PFPE), polyurethane, polyimides, and cross-linked NOVOLAC® (phenol formaldehyde polymer) resins, glass (including, but not limited to, borosilicate glass, SF11, and SF12), quartz, cyclic olefin copolymers (COC), cyclic olefin polymers (COP), acrylate polymers, polystyrene and polycarbonate. In certain embodiments the system further comprises comprising a pump or pressure system (or gravity feed system, or electrokinetic system) to move cells and/or reagents through or into the microchannels and/or the microcavities. In certain embodiments the imaging system comprises a digital camera (e.g. a CCD camera). In certain embodiments the imaging system comprises a microscope objective. In certain embodiments the microfluidic device is configured on a movable stage to move the device with respect the microscope objective so that different microcavities can be imaged by the same objective. In certain embodiments the microscope objective can be moved with respect to the microfluidic device to permit alignment of the objective with different microcavities. In certain embodiments the system further comprises one or more detection reagents to label radiation induced foci in cells. In certain embodiments the detection reagents comprise labeled antibodies that bind to radiation induced foci. In certain embodiments the antibodies are selected from the group consisting of anti-P53 binding protein 1, anti-γH2AX, anti-Rad51, anti-MRE11, anti-XRCC1, anti-Rad50, anti-BRCA1, anti-ATM, anti-ATR, and anti-DNApkcs. In various embodiments the system is operably connected to a computer. In certain embodiments the computer is configured to quantify radiation-induced foci in images acquired by the imaging system. In certain embodiments the computer is configured to determine a repair kinetic for radiation induced foci (RIF) using a model where one double strand break (DSB) is detected at a rate k₁ leading to the formation of one RIF and one RIF is resolved after repair at rate k₂ assuming that both processes are irreversible where the model can be expressed by the equations shown as Eq 1 herein. In certain embodiments the computer is further configured to perform one or more actions selected from the group consisting of operating the image analysis system to capture an image, adjusting the field location and/or focus of the microscope objective, determining the location of cells and/or cellular nuclei within an acquired image, controlling the passage of cells and/or reagents into and/or through the microfluidic device.

In various embodiments methods of determining the sensitivity of a subject to ionizing radiation and/or to non-ionizing radiation and/or the risk of adverse consequences of the radiation to the a subject are provided. The method typically involves contacting a biological sample comprising cells from the subject with ionizing or non-ionizing radiation; detecting and quantifying radiation induced foci in the cells at least two different time points; and determining a repair kinetic for the radiation induced foci that is a measure of the rate of disappearance of the foci, where a longer repair kinetic indicates a greater sensitivity of the subject to radiation. In certain embodiments the contacting comprises contacting the sample to ionizing radiation. In certain embodiments the ionizing radiation is produced by a radionuclide or by an x-ray source. In certain embodiments the contacting comprises contacting the sample to non-ionizing radiation. In certain embodiments the non-ionizing radiation source is a UV source. In certain embodiments high dose radiation is used and the repair kinetic provides a measure of acute response to radiation. In certain embodiments high dose and low dose radiation is used and the repair kinetic provides a measure of cancer risk. In certain embodiments the contacting, detecting, and determining is performed using a system as described herein. In certain embodiments the repair kinetic for radiation induced foci (RIF) is determined using a model where one double strand break (DSB) is detected at a rate k₁ leading to the formation of one RIF and one RIF is resolved after repair at rate k₂ assuming that both processes are irreversible where the model can be expressed by Eq. 1 shown herein. In certain embodiments the repair kinetic is evaluated with respect to the same kinetic determined for the subject at an earlier time and an increase in the kinetic indicates increasing radiation susceptibility of the subject over time. In certain embodiments the repair kinetic is evaluated with respect to the same kinetic determined for a population or subpopulation and a repair kinetic longer than the average or median repair kinetic for the population or subpopulation indicates that the subject has elevated radiation sensitivity and a repair kinetic shorter than the average or median repair kinetic for the population or subpopulation indicates that the subject has reduced radiation sensitivity. In certain embodiments α (in Eq. 1) alpha reflects DSB clustering and the lower alpha the higher the risk. In certain embodiments sensitivity or risk is identified at two different radiation doses, where the different sensitivity or risk determined at each dose provides a measure of sensitivity or risk for low dose exposures and for high dose exposures. In certain embodiments the repair kinetic is normalized to an average or to a median value for a population or subpopulation. In certain embodiments the repair kinetic is normalized to a subpopulation and the subpopulation comprises members grouped/selected by one or more factors selected from the group consisting of ethnicity, age, gender, occupation, and disease state. In certain embodiments the cells comprise cells selected from the group consisting of erythrocytes, lymphocytes, primary cells from biopsies. In certain embodiments the cells are cells from a human (e.g., a human that is to be subjected to radiotherapy and/or medical imaging, and/or a human that works in a region subject to radiation risk). In certain embodiments the cells are cells from a non-human mammal (e.g., a non-human primate, a canine, a feline, a bovine, an equine, a porcine, a lagomorph, and the like). In certain embodiments the repair kinetic and/or a diagnosis/prognosis based, at least in part, on the repair kinetic is recorded in a patient medical record. In certain embodiments the patient medical record is maintained by a laboratory, physician's office, a hospital, a health maintenance organization, an insurance company, or a personal medical record website. In certain embodiments the repair kinetic and/or a diagnosis/prognosis based, at least in part, on the repair kinetic is recorded on or in a medic alert article selected from a card, worn article, or radiofrequency identification (RFID) tag. In certain embodiments the repair kinetic and/or a diagnosis/prognosis based, at least in part, on the repair kinetic is recorded on a non-transient computer readable medium. In certain embodiments when the measure indicates a heightened radiation sensitivity of the subject, as compared to a reference population, adjusting life style and dietary habits as preventive measures.

In certain embodiments a method of determining the sensitivity of a subject to ionizing radiation and/or to non-ionizing radiation and/or risk of adverse consequences of said radiation to said a subject is provided where the method comprises providing a biological sample from the subject comprising cells; and detecting and quantifying baseline foci in the cells to provide a foci number; where an increase in foci number as compared to a reference foci number determined for said subject at a previous time or for a population indicates elevated sensitivity of a subject to ionizing radiation and/or to non-ionizing radiation and/or risk of adverse consequences of said radiation to said subject and a decrease in foci number as compared to a reference foci number determined for said subject at a previous time or for a population indicates decreased sensitivity of said subject to ionizing radiation and/or to non-ionizing radiation and/or risk of adverse consequences of said radiation to said subject. In certain embodiments the foci number is evaluated with respect to the same foci number determined for said subject at an earlier time and an increase in said foci number indicates increasing radiation susceptibility of said subject over time. In certain embodiments the foci number is evaluated with respect to the same foci number determined for a population or subpopulation and a foci number larger than the average or median foci number for said population or subpopulation indicates that said subject has elevated radiation sensitivity and a foci number lower than the average or median foci number for said population or subpopulation indicates that said subject has reduced radiation sensitivity. In certain embodiments foci number is normalized to an average or to a median value for a population or subpopulation. In certain embodiments the foci number is normalized to a subpopulation and said subpopulation comprises members grouped/selected by one or more factors selected from the group consisting of ethnicity, age, gender, occupation, and disease state. In certain embodiments the sample comprises whole blood, or a blood fraction. In certain embodiments the sample comprises cells selected from the group consisting of erythrocytes, lymphocytes, primary cells from biopsies. In certain embodiments the sample/cells are from a human (e.g., a human that is to be subjected to radiotherapy and/or medical imaging, a human that works in a region subject to radiation risk, and the like). In certain embodiments the cells are cells from a non-human mammal (e.g., a non-human primate, a canine, a feline, a bovine, an equine, a porcine, a lagomorph, etc.). In certain embodiments the foci number and/or a diagnosis/prognosis based, at least in part, on the foci number is recorded in a patient medical record. In certain embodiments the patient medical record is maintained by a laboratory, physician's office, a hospital, a health maintenance organization, an insurance company, or a personal medical record website. In certain embodiments the foci number and/or a diagnosis/prognosis based, at least in part, on said foci number is recorded on or in a medic alert article selected from a card, worn article, or radiofrequency identification (RFID) tag. In certain embodiments the foci number and/or a diagnosis/prognosis based, at least in part, on said foci number is recorded on a non-transient computer readable medium. In certain embodiments when the measure indicates a heightened radiation sensitivity of the subject, as compared to a reference population, life style and dietary habits are adjusted as preventive measures. In certain embodiments the detecting and quantifying is performed using a system comprising a microfluidics device comprising one or a plurality of microfluidic cavities each configured to contain cells; and an imaging system configured to detect radiation-induced foci in said cells when they are disposed in said one or plurality of microfluidic cavities. In certain embodiments the microfluidic device comprises at least one, or at least two, or at least four, or at least eight microcavity cells for each sensitivity determination that is to be performed. In certain embodiments the microfluidic device is operably coupled to or further comprises a cell separator. In certain embodiments the cell separator is configured to separate lymphocytes from a blood or blood fraction sample and deliver said lymphocytes into the microfluidic cavities. In certain embodiments the channels or chambers in said cell separator are coupled to said microcavities by microchannels and configured to deliver said lymphocytes from said separator into said microcavities. In certain embodiments the device lyses erythrocytes and isolates leukocytes. In certain embodiments the microfluidics device comprises a fabricated block within which are formed, embedded or molded, one or more fluid-tight channels. In certain embodiments the block material from which the device is fabricated is selected from the group consisting of polydimethylsiloxane (PDMS), polyolefin plastomer (POP), perfluoropolyethylene (PFPE), polyurethane, polyimides, and cross-linked NOVOLAC® (phenol formaldehyde polymer) resins, glass (including, but not limited to, borosilicate glass, SF11, and SF12), quartz, cyclic olefin copolymers (COC), cyclic olefin polymers (COP), acrylate polymers, polystyrene and polycarbonate. In certain embodiments the device/system comprises a pump or pressure system to move cells and/or reagents through or into the microchannels and/or the microcavities. In certain embodiments the imaging system comprises a digital camera or camera chip. In certain embodiments the imaging system comprises a microscope objective. In certain embodiments the device comprises one or more detection reagents to label radiation induced foci in cells. In certain embodiments the detection reagents comprise labeled antibodies that bind to radiation induced foci. In certain embodiments the antibodies are selected from the group consisting of anti-P53 binding protein 1, anti-γH2AX, anti-Rad51, anti-MRE11, anti-XRCC1, anti-Rad50, anti-BRCA1, anti-ATM, anti-ATR, and anti-DNApkcs. In certain embodiments the system is operably connected to a computer. In certain embodiments the computer is configured to foci in images acquired by said imaging system. In certain embodiments the computer is configured to perform one or more actions selected from the group consisting of operating said image analysis system to capture an image, adjusting the field location and/or focus of said microscope objective, determining the location of cells and/or cellular nuclei within an acquired image, controlling the passage of cells and/or reagents into and/or through said microfluidic device.

In various embodiments methods of administering radiation therapy to a subject and/or imaging the subject are provided. The methods typically involve receiving a measure of sensitivity to radiation based on a measurement of a sample from the subject as described herein; and where, when the measure indicates a heightened radiation sensitivity of the subject, as compared to a reference population, adjusting the mode of administration of the radiotherapy reduce off-target radiation exposure, and/or to increase recovery times between periods of radiation administration; and/or where, when the measure indicates a heightened radiation sensitivity of the subject, as compared to a reference population, adjusting the imaging modality to reduce exposure to ionizing radiation. In certain embodiments the method comprises a method of administering radiation therapy to a subject and, when the measure indicates a heightened radiation sensitivity of the subject, as compared to a reference population, the mode of administration of the radiotherapy is adjusted to reduce off-target radiation exposure, and/or to increase recovery times between periods of radiation administration. In certain embodiments the radiation therapy comprises application of external radiation and the administration is adjusted by increasing the number of exposure directions to improve skin sparing. In certain embodiments the radiation therapy comprises application of internal radiation and the administration is adjusted by utilizing radioisotope that have a shorter half-life and/or that are lower energy. In certain embodiments the administration is adjusted by increasing recovery times between rounds of administration. In certain embodiments the method comprises a method of medical imaging in the subject and, when the measure indicates a heightened radiation sensitivity of the subject, as compared to a reference population, the imaging modality is adjusted to reduce exposure to ionizing radiation. In certain embodiments the imaging modality is adjusted by utilizing NMR or ultrasound. In various embodiments the subject is a human or a non-human mammal.

In various embodiments methods of evaluating cancer risk in a subject are provided. The methods typically involve receiving a measure of sensitivity to radiation based on a measurement of a sample from the subject according to the methods described herein; and where, when the measure indicates a heightened radiation sensitivity of the subject, as compared to a reference population, the subject is identified as at elevated risk for cancer. In certain embodiments when the measure indicates a heightened cancer risk of the subject, as compared to a reference population, the life style and dietary habits are adjusted as preventive measures. In certain embodiments the measure of sensitivity to radiation, or a cancer risk based, at least in part, on measure of sensitivity to radiation, is recorded in a patient medical record. In certain embodiments the patient medical record is maintained by a laboratory, physician's office, a hospital, a health maintenance organization, an insurance company, or a personal medical record website. In certain embodiments the measure of sensitivity to radiation, or a cancer risk based, at least in part, on measure of sensitivity to radiation, is recorded on or in a medic alert article selected from a card, worn article, or radiofrequency identification (RFID) tag. In certain embodiments the measure of sensitivity to radiation, or a cancer risk based, at least in part, on measure of sensitivity to radiation, is recorded on a non-transient computer readable medium.

### DEFINITIONS

The terms “microfluid channel” or “microfluidic channel” are used interchangeably to refer to a channel that has a characteristic dimension (e.g., width and/or depth) about 500 microns or less. In certain embodiments the characteristic dimension ranges from about 1, 5, 10, 15, 20, 25, 35, 50 or 100 microns up to about 150, 200, 250, 300, or 400 microns. Typical microfluidic channels have dimensions sufficient to allow passage of a mammalian cell.

A “microfluid cavity or chamber” or “microfluidic cavity” or “microfluidic chamber” refers to chamber or cavity that has a characteristic dimension (e.g., width and/or depth) about 500 microns or less. In certain embodiments the characteristic dimension ranges from about 1, 5, 10, 15, 20, 25, 35, 50 or 100 microns up to about 150, 200, 250, 300, or 400 microns. Typical microfluid chambers have dimensions sufficient to allow contain a plurality of mammalian cells.

The terms microfluidic device” and “microfluid device” are used interchangeably to refer to devices comprising one or more microfluid chambers and/or channels. Typically microfluidic devices are configured to permit that transfer of materials (fluids, cells, etc.) into and/or through one or more microfluid channels and/or chambers comprising the device. In various embodiments microfluidic devices permit the transport and/or manipulation of volumes of fluid on the order of nanoliters or picoliters.

The term “subject” and “patient” are used interchangeably to refer to a mammal from which a biological sample is obtained to determine sensitivity to ionizing and/or non-ionizing radiation. Subjects can include humans and non-human mammals (e.g., a non-human primate, canine, equine, feline, porcine, bovine, lagomorph, and the like).

The term “biological sample” or “test sample” refers to sample is a derived from a subject that, in the present case, comprises mammalian cells containing cell nuclei and nuclear DNA. Such samples include samples from humans and non-human mammals, sample of biological fluids that contain cells (e.g., blood samples) and samples from various tissues. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, inactivation of interfering components, the addition of reagents, lysing, etc. Such “treated” or “processed” samples are still considered to be biological samples with respect to the methods described herein.

### BRIEF DESCRIPTION OF THE DRAWINGS

FIG. 1 illustrates the correlation between DNA repair and radiation toxicity. DNA repair kinetic was obtained by applying the mathematical algorithm shown in the Examples to radiation induced foci kinetic reported in Rübe et al. (2008) Clin. Cancer Res, 14: 6546-6555, for four different breeds of mice (CB57: normal breed, Balb/C: sensitive breed, SCID: immunodeficient mice, and AT: DNA repair compromised mice). The death toxicity was obtained from various LD50 measurements published in the literature (LD50 is the minimum dose necessary to kill 50% of the mice).

FIGS. 2A-2D illustrate one system used for the measurements described herein. FIG. 2A schematically illustrates one microfluidic device (chip) used for the measurement. Each chamber can accommodate six irradiation spots. FIGS. 2B and 2C illustrate a prototype setup for an X-ray focused-beam system. FIG. 2B illustrates the overall setup, with the small X-ray device mounted on motorized micromanipulator, allowing precise positioning of beam. FIG. 2C shows the beam nozzle oriented towards a specimen. FIG. 2D shows a large field of view (5× magnification), with γH2AX immunostaining to visualize irradiated area (4 mm collimation). Using higher magnification, one can simultaneously measure damage (bright spots) in cells in S-phase (red staining—Click-IT, Invitrogen), G1 and G2.

FIG. 3 shows an illustrative flow diagram of sample processing in seven steps. Step 1: Pump blood inside LOC; Step 2: Sort Lymphocytes and discard red blood cells; Step 3: Irradiate Lymphocytes; Step 4: Incubate lymphocytes, fixed with 4% Paraformaldehyde at specific times and label cell for DNA damage (i.e. p53 binding protein 1-53BP1); Step 5: Automatic acquisition of images for labeled microcavities; Step 6: Automatic image analysis leading to nuclear segmentation and spot counting; Step 7: Collect number of RIF/cell for each time point and generate repair curve to compute radiation sensitivity risk factor (i.e. Rad Blood Type).

FIG. 4 is a block diagram showing an illustrative example of a logic device in which various aspects of the methods and systems described herein may be embodied.

FIGS. 5A-5C, illustrate time-lapse imaging of MCF10A transiently transfected with 53BP1-GFP after exposure to 0.1 Gy of X-rays. FIG. 5A: Representative snapshots of best focal plane for a 3D time lapse. Counting was done manually in two different ways: (i) static measurement, indicating the number of RIF/cell at the time it is measured (bottom graphs); (ii) cumulated measurement, indicating at any time the overall number of different RIF that have appeared since time 0 (top graphs). The 53BP1 nuclear bodies visible before IR were not included in RIF counts. FIG. 5B: RIF counts from 40 different time lapses (three independent experiments) leads to an average for T_{1/2induction}=15 min, T_{1/2resolution}=1.4 h, α=73 RIF/Gy. Fits are shown as solid lines, and experimental points as square for cumulated counts and triangles for net counts (R²=0.98 and t test P value=0.005 for the fit). FIG. 5C: One-dimensional intensity profiles of four different regions of interest indicated by blue dash box in A. The average profile is indicated by solid curve and used to evaluate the average size of a focus (defined as the full width at half maximum of the peak).

FIGS. 6A-6C, illustrate time-lapse imaging of MCF10A transiently transfected with 53BP1-GFP after exposure to 1 Gy of X-rays. FIG. 6A: representative snapshots of best focal plane for a 3D time lapse. FIG. 6B: RIF counts from 21 different time lapses (three independent experiments) leads to an average for T_{1/2induction}=6.5 min, T_{1/2resolution}=2.1 h, α=28 RIF/Gy, R²=0.99, and t test P value=0.003 for the fit. FIG. 6C: One-dimensional intensity profiles of five different regions of interest indicated by blue dash box in FIG. 6A.

FIG. 7, panels A-F, illustrates representative time response of background corrected RIF per nucleus in MCF10A exposed to various doses of X-rays and immunostained for 53BP1. Panels A, B, and C: Maximum intensity projections of representative 3D stack images at various doses (red dots indicate detected 53BP1 RIF) are accompanied with the same nucleus overlaid with the full shape identification of an RIF and the ability of the algorithm to separate touching RIF even at the maximum dose (panel C −2 Gy, see enlargement). In these images, each RIF is labeled by the algorithm with a different color to facilitate individual visualization. Panel D: Time response under normal media conditions for one experiment out of seven performed (average R² across all doses is 0.98 and t test P value is less than 0.01). Experimental data points (circles) get larger with dose (0.1, 0.4, 1, and 2 Gy, respectively), and they correspond to averages for approximately 1,000 nuclei per point, with their corresponding standard deviations. Solid lines are least-square fits using Eq. 1 for each time response. Panel E: Inhibition of ATM measured by Western blot of P53-S15p. Panel F: Time response under ATM inhibition (average R² across all doses is 0.99 and t test P value is less than 0.01), based on one experiment.

FIG. 8, panels A-C, show representative time response of background corrected RIF per nucleus in MCF10A exposed to 1 GeV/atomic mass unit Fe ions and immunostained for 53BP1. Panel A: Representative images for 1.5 and 30 min post-IR, illustrating which RIF are classified “core RIF” vs “delta-ray RIF”. Panel B: Time response averaged over five independent experiments cumulating more than 1,000 tracks per time point and experiment. Delta-ray RIF are reported as RIF/nucleus per Gy (red), whereas core RIF are reported as RIF/μm (blue). Panel C: Average normalized intensity profiles at 1.5 and 30 min post-IR for core and delta-ray RIF (N=20 for each profile—RIF diameters are shown as the full width at half maximum of the peaks).

FIG. 9, panels A-C, shows average fitted parameters for all time responses measured in human MCF10A. Four conditions are considered (fixed, normal condition, immunostaining of 53BP1, N=7; fixed-ATM, ATM inhibition and immunostaining of 53BP1, N=1; fixed-Fe, 53BP1 immunostaining after exposure to 1 GeV/atomic mass unit Fe, with estimated doses along ion tracks of 26 Gy and outside tracks of 0.17 Gy, N=5; live, time-lapse imaging of MCF10A transiently transfected with 53BP1-GFP after exposure to 0.1 and 1 Gy of X-rays, N=3). All trends are statistically significant with respect to dose using one-way ANOVA test (P<0.01 for a and P<0.05 for k₁ and k₂). Statistical differences between dose points are tested with the Tukey-Kramer test and are indicated by an asterisk with the color corresponding to the group when significant. Panel A: Absolute RIF yield α (RIF/Gy per nucleus), showing a decrease with dose. Panel B: RIF induction half-life (ln(2)/k₁), showing a faster induction with dose. Panel C: (C) RIF disappearance-resolution half-life (ln(2)/k₂) showing a slower RIF resolution with dose.

FIG. 10 shows time-lapse imaging of human fibrosarcoma HT1080 stably transfected with 53BP1-GFP. (Upper) Representative snapshots of movies for three different doses (5, 10, and 100 cGy). Counting was done manually and done in two different ways: (i) static measurement, indicating the number of RIF/cell at the time it is measured (numbers and graphs); (ii) cumulated measurement, indicating at any time the overall number of different RIF that have appeared since time 0 (red numbers and graphs). (Lower) The average of these counts from 20-40 nuclei per dose (square for cumulated averages and triangles for static averages). Static and cumulated averages could be fitted simultaneously with the same parameters using Eqs. 1 and 2, respectively.

FIG. 11 illustrates human validation of spot detection algorithm. Human mammary epithelial cells (MCF10A) were exposed to various doses of X-rays, immunostained for 53BP1, and RIF were counted manually or by algorithm from 3D stacks. Graph plotting RIF counts scored by computer algorithm against RIF counts scored blindly by human eye for various doses show good agreement. A total of 350 nuclei were scored here, and each nuclear count is represented as a circle, with circles of larger sizes for larger doses. Linear regression led to an overall R²=0.88 and P value for t test less than 0.05 (lower graph), indicating good agreement between manual and automatic counts.

FIG. 12, panels A-C, illustrate RIF dose-kinetic fits from single experiment performed on normal human skin diploid fibroblasts in G1 (HCA2) imaged by 3D microscopy and stained for 53BP1. Panel A: Absolute RIF yield a (RIF/Gy per nucleus), showing a decrease with dose. Panel B: RIF induction kinetics constants (0, showing a faster induction with dose. Panel C: RIF disappearance-resolution kinetics constants (k₂) showing a slower RIF resolution with dose. Note that because only one experiment was performed here, error bars represent standard deviations from measurements made over 3,000 nuclei per dose point. Trend significance using t test between [0.1, 0.4] Gy group and high dose group and using duplicate well as separate measurements (P<0.05) are shown by asterisk.

FIG. 13 shows an illustration of energy deposition from HZE particle 1 GeV/atomic mass unit Fe ion in a theoretical rectangular cell. A small red cylinder crossing the cell in the middle along its length indicates the core of the HZE. Delta rays generated in the core via Coulomb interactions are depicted as wavy arrows.

FIG. 14 shows time-lapse imaging of MCF10A exposed to 1 track of 1 GeV/atomic mass unit Fe ion (approximately 0.24 Gy, LET˜148 keV/μm). Cells are transiently transfected with 53BP1-GFP RIF and H3-dsRed. Time-lapse confirms delayed kinetics for the apparition of low-LET RIF (appearing delta-rays RIF are indicated by blue arrows in each time frame). Track RIF frequency here is approximately 0.65 RIF/μm across the time points 11 to 30 min post-IR, whereas low-LET RIF frequencies reach a maximum between 24 and 30 min post-IR.

FIG. 15, panel A, shows Stably transfected human bronchial epithelial cells (HBEC) exposed to 1 track of 1 GeV/atomic mass unit 0 ions (approximately 0.022 Gy, LET˜14 keV/μm). FIG. 15, panel B, shows that control HBEC, that did not get irradiated, show no induction of foci for similar time-lapse acquisition frequency. This confirms that delayed foci appearing in panel A are not the result of photodamage from imaging.

FIG. 16, panels A-F, shows immunofluorescence (IF) staining optimization. Optimization of 53BP1 staining was performed for the concentrations and incubation times of three parameters: (i) the blocking agent (BSA), (ii) the primary antibody (1° Ab), and (iii) the secondary antibody (2° Ab). The unoptimized staining protocol was blocking with 0.1% BSA (1 h), incubation with 1° Ab (1:200, 1 h) and 2° Ab (1:200, 2 h). Panel A: IF intensities of 53BP1 staining for various incubation times of the 1° and 2° Ab (using optimized Ab concentrations). The relative foci intensities saturate for all conditions after about 16 min. This indicates short incubation times (approximately 15-20 min), with higher concentrations of Ab (1:100) is enough for optimum results. This conclusion is supported by visual analysis of the microscopic images, as is the effect of combining each optimized parameter, which appears additive (Panels B-E). Panel F: Picture of an ampligrid slide (reprinted, with permission from Beckman Coulter).

FIG. 17, panels A-E, shows impact of foci sizes on detection. Panel A: Distribution of 53BP1 RIF volumes after 2 Gy of X-rays in MCF10A. Panel B: Representative MIP is shown below the graph. The average focus volume for this time point is 0.45 μm³. The distribution indicates that 95% of RIF have volumes lower than 1 μm³. Panels C-E: Using the same set of nuclei, random spots were generated in the 3D volumes defined by each nucleus from an average of 1 to 40 foci/nucleus. The same spots were expanded using a Gaussian filter with various sigma values (α=0, 1, 2, 3, 4) leading to various foci volumes (0.1, 0.4, 1.3, and 2.4 μm³, respectively). The position of each of this foci volumes are depicted with the same color on the distribution graph in panel A—except 2.4 μm³, which is off the chart. Panel C: For 1.3 and 2.4 μm³ foci, detection is statistically lower than reality when the average number of foci/nucleus is greater than 30 and 20, respectively. Panel D: The reported foci volume as a function of the number of foci/nucleus for different simulated foci volumes. One can note that the algorithm can maintain accurate count by reducing the reported volume of the foci. Panel E: MIP of the corresponding images for these different expansions.

FIG. 18 illustrates background correction. The number of RIF/nucleus for each time point following two doses (0.15 and 2 Gy) in MCF10A labeled with 53BP1 were corrected for background level of background foci. Count distribution for RIF/nucleus are shown as histogram [H(Dose)] and fitted by a Poisson distribution of mean M(POIS(M)) convolved with the foci/nucleus distribution of unirradiated specimen (curve, Top, H(0,Gy)). The mean M that led to the best fit, which is displayed over each histogram as a blue solid line, corresponds to the reported real RIF yield for a given time point corrected for background foci. As one would expect, these graphs confirm that the number of real RIF/nucleus follow a Poisson distribution much like the number of DSB/nucleus.

### DETAILED DESCRIPTION

In various embodiments methods and devices for identifying the sensitivity to radiation of a biological sample (and by implication for the subject from whom the sample is derived) are provided. The methods and devices permit the rapid and efficient determination of sensitivity to radiation. Such measurements of radiation sensitivity for individuals have numerous uses.

For example, in certain embodiments, such measurements can be used in radiotherapy. Using such an assay, permits prediction/identification of subjects that are likely to have an acute reaction to repeated exposures of high levels of ionizing (or non-ionizing) radiation. In case of a predicted sensitivity, a modified therapy could be proposed and administered to the patient. For example, one could reduce the total dose per session and increase the numbers of sessions (e.g., hyperfractionated radiotherapy), and/or one could increase the recovery period between sessions, and/or one could distribute the entrance paths (e.g., for external radiation sources) to improve skin sparing.

In medical imaging if a subject is determined to be sensitive to ionizing radiation, the information would allow a patient, and/or a doctor, and/or an insurance plan to justify the usage of medical devices or therapy that do not involve ionizing radiation (e.g. MRI, ultrasound, chemotherapy, etc.).

The methods and devices described herein also find use in monitoring subjects occupationally exposed to radiation sources. Employers could constrain radiation sensitive employees to a lower annual exposure limit. For example, the maximum limit of ionizing radiation is 1000 mrem/year for regular employees at LBNL. However pregnant women are considered sensitive employees with an annual limit of 500 mrem.

The methods and devices described herein can also be used to identify at risk subjects in a population subject to possible environmental exposure from a radiation source (e.g., in the instance of a nuclear plant failure or material release), etc.

In addition, by describing the average response across hundreds of subjects (e.g., human blood donors), one can characterize the repair kinetic of a full population (or subpopulation). An individual donor's measurement can then be normalized to the population/subpopulation (e.g., the individual donor's repair kinetic constant could be divided by the average or median repair kinetic constant for the population), leading to a final risk score. In this example, (individual metric/population average metric) a value below one would indicate resistance to radiation and a value above one would indicate radiation sensitivity. This method can be put in place in hospital, permitting linkage of this risk factor to specific medical short term endpoints (e.g., skin sensitivity to radiotherapy) and by establishing a clear monitoring program, e.g., with the SFA low dose program of the DOE, one use such scores for cancer risk assessment.

The methods and devices described herein utilize the quantification of biological markers of DNA damage in cells (e.g., human or non-human mammal cells) to provide a measure radiation sensitivity. It has been shown that most DNA containing cells in a mammal respond similarly to ionizing radiation. In particular double strand DNA breaks (DSBs) are the most deleterious form of radiation-induced DNA damage, and it is believed that DSB repair deficiencies can lead to radiosensitivity (see, Rübe et al. (2008) Clin. Cancer Res, 14: 6546-6555).

Without being bound to a particular theory, it is believed that sample cells (e.g., blood leukocytes) can be used as a surrogate system to evaluate the response of a person (or non-human mammal) to ionizing radiation.

Functional assays are described herein that unambiguously characterize the efficiency of DNA repair at the individual level, by quantifying DNA damage foci (e.g., 53BP1 foci) kinetics. This technology is much simpler than previous approaches and can be scaled up to provide an inexpensive and rapid assay allowing its deployment in various medical and work environments.

In various embodiments the assays contemplated herein involve providing a biological sample from a subject (e.g., a small blood sample from a person) and the sensitivity to radiation (e.g., ionizing radiation and/or non-ionizing radiation) of that subject is determined by computing a DNA repair kinetic (e.g., as described herein in the Examples (see, also, Neumaier et al. (2012) Proc. Natl. Acad. Sci., U.S.A., 109(2): 443-448).

Basically, in various embodiments, a simple mathematical model is provided describing radiation induced foci (RIF) formation where one DSB is detected at a rate k₁ leading to one RIF, and one RIF is resolved after repair at a rate k₂ assuming both processes are irreversible. This model can be noted as follows:

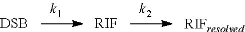

where C₀ and C₁ are the average number of DSB and RIF per nucleus at time t, respectively. This kinetic model translates then into the following set of differential equations:

$$\{ \begin{matrix}\frac{d {C}_{0}}{d t} = - {k}_{1} {C}_{0} \\ \frac{d {C}_{1}}{d t} = {k}_{1} {C}_{0} - {k}_{2} {C}_{1}\end{matrix} {⇒}^{{C}_{1} ( 0 ) = 0} \{ \begin{matrix}{C}_{0} ( t ) = α D . {e}^{- {k}_{1} t} \\ {C}_{1} ( t ) = \frac{α {Dk}_{1}}{{k}_{2} - {k}_{1}} ( {e}^{- {k}_{1} t} - {e}^{- {k}_{2} t} )\end{matrix} \qquad [ 1 ]$$

where α is the number of naked DSB/Gy before formation of RIF and D is the dose delivered to the cell. Alpha (α) is preferably be constant for all doses. Further details are provided in the Examples regarding the way Eq. 1 is fitted.

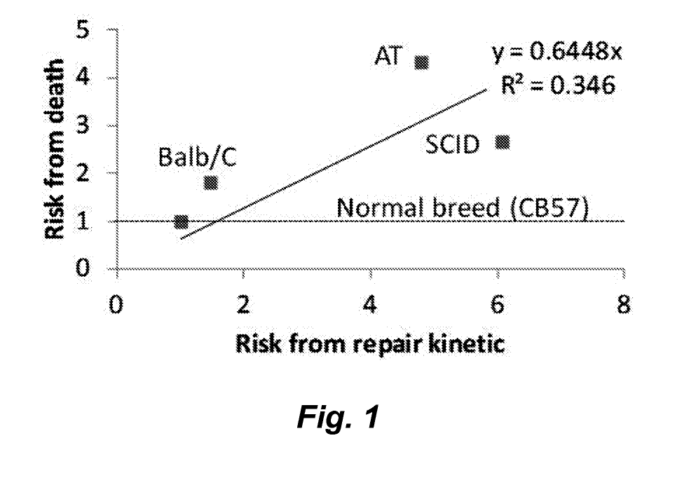

In certain embodiments, the repair kinetic constant is normalized to an average or median baseline determined for a population. In such instances, if the repair kinetic constant is close to the average baseline measured in a representative population nor subpopulation (to be determined), the subject's risk factor is 1 (no risk). On the other hand, if the repair kinetic is N times slower, then the subject's risk factor is N indicated greater radiation sensitivity and/or increased health risk associated with exposure to radiation. FIG. 1 shows that risk factors computed this way correlate well with radiation sensitivity measured in four different breeds of mice of varying resistance to radiation.

In various embodiments sensitivity to radiation and/or risk can also evaluated by another factor, designated alpha (α) in equation 1, that reflects DSB clustering. A lower The lower alpha, the higher the clustering, the higher the risk. Also, the assays described herein determine a risk that is dose dependent, so two risk factors can be computed: one at low dose and one at high dose. Again, this is reflected by alpha and the fact that alpha goes up with dose. This dose dependence may not be the same in different individuals and therefore there relative risk to radiation may be different for high and low radiation dosages.

By miniaturizing the assay, e.g., by using a mini X-ray source, by keeping/analyzing cells in small microfluidics devices, and by providing integrated analytical tools, it is possible to process multiple samples (e.g., blood samples) within, e.g., 4, 8, or 12 hours. The time delay (e.g., 12 hour delay permits evaluation of the rapidity with which the cells (e.g., lymphocytes) repair DNA damage).

Accordingly, in certain embodiments, the assay can be automated by configuring a small X-ray device (or other source of ionizing or non-ionizing radiation) to irradiate a microchannel or microchamber containing sample cells (e.g., lymphocyte cells from human blood). In certain embodiments the cells are flowed through one or more microchannels, where the flow rate and radiation source can determine radiation exposure (e.g. fast flow rate for small doses). These cells are then incubated inside microcavities, e.g., in the microfluidic device, and then immunostained for double strand breaks (DSBs) using for example an antibody that binds to regions characterized by DSBs e.g., anti-p53 binding protein 1, 53BP 1; anti-γH2AX, anti-Rad51, anti-MRE11, anti-XRCC1, anti-Rad50, anti-BRCA1, anti-ATM, anti-ATR, anti-DNApkcs, and the like).

This results in nuclear images with small circular spots (foci) marking DNA DSBs. The kinetic at which these foci disappear can be linked to radiation sensitivity. For example, applying the mathematical approach described herein (see, Examples) to published foci data, we can show that repair kinetic correlate well with radiation sensitivity. More specifically SCID mice are 4.3 times more likely to die from an acute dose of IR than the resistant breed CB57. Similarly, the repair kinetic of SCID mice measured by foci kinetic is 4.8 times slower.

Image acquisition can be performed using for example, a small microscopic device and analysis of the foci can be performed using automated software. In certain embodiments the microfluidic device can further incorporate or can be operably linked to other devices that facilitate sample processing (e.g., separating erythrocytes from lymphocytes in a blood sample).

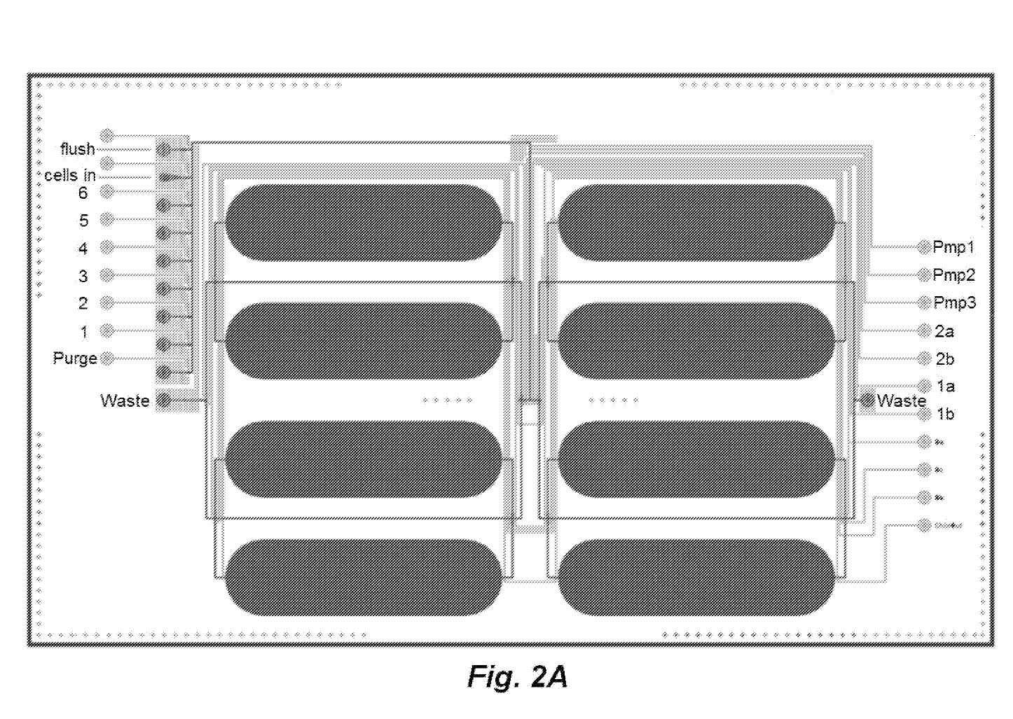

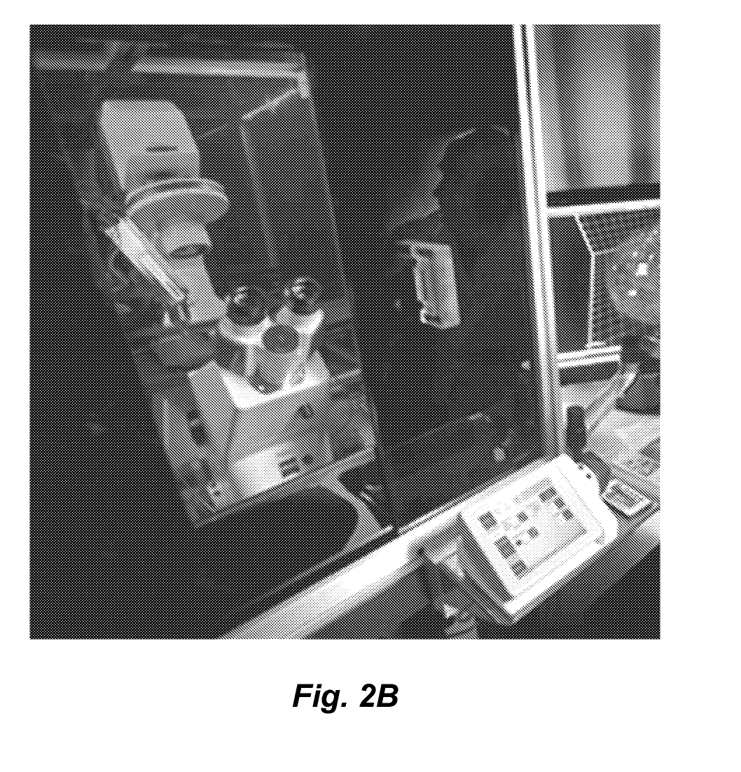

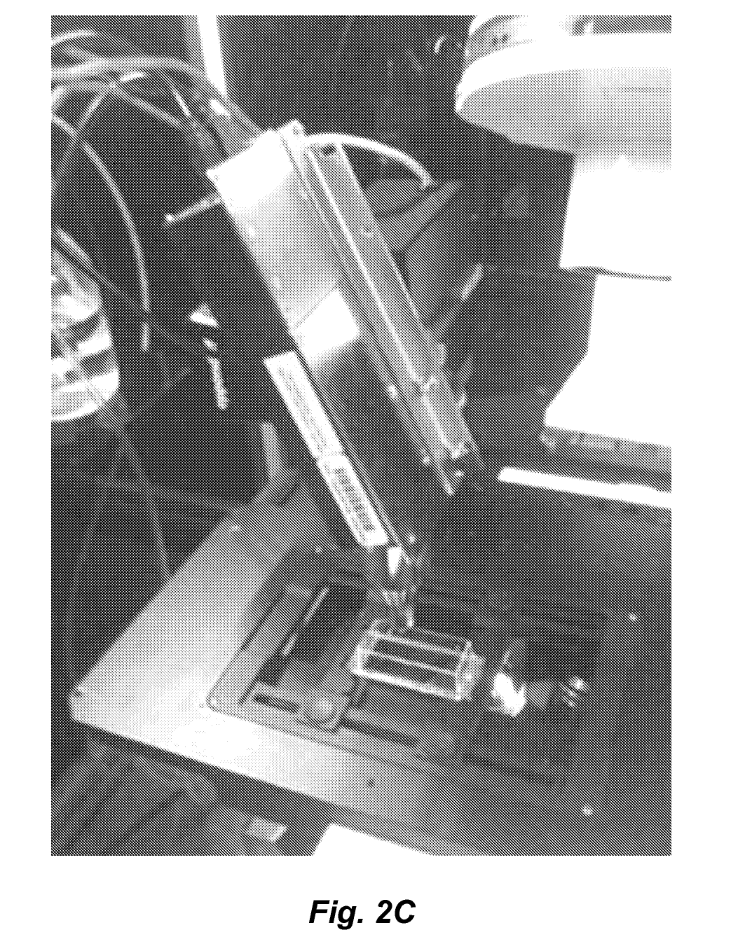

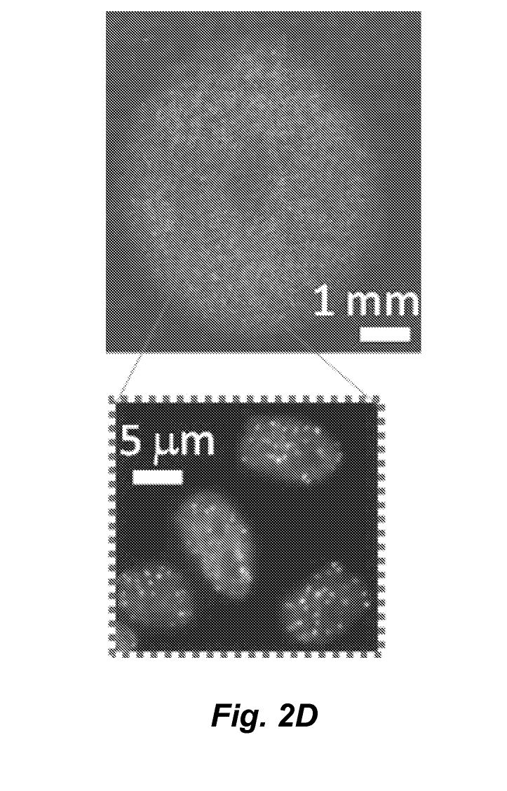

In one illustrative, but not limiting system, a miniaturized X-ray tube (e.g., MiniX, Amptek, Inc.) and an engineered Lab On a Chip (LOC), is utilized (see, e.g., FIGS. 2A 2B). This device has been used to image human breast cells exposed to ionizing radiation and fixed 1 to 24 hours after exposure (see, FIG. 2D). The illustrated chip (microfluidic device) comprises 8 chambers, where each chamber can accommodate ˜6 irradiation spots (see FIG. 2A). Upon irradiation, small nuclear domains are formed around the DNA damage sites, and can be labeled with fluorescent antibodies (e.g., phosphorylated histone H2AX-γH2AX, or p53 binding protein 1-53BP1). These spots are referred as radiation induced foci (RIF) and are illustrated in FIG. 2D. An image algorithm and mathematical fitting techniques to extract from RIF kinetic, accurate repair kinetic as described in the examples.

In the device illustrated in FIG. 2, image acquisition was done with a commercial microscope (Carl Zeiss AxioObserver) with a motorized stage allowing automatic scanning and imaging of different microcavities where cells are incubated for different time post-irradiation. Cell chemical fixation was done with 4% paraformaldehyde and immunostaining was also done automatically by a computer-controlled pump system in the lab-on-a-chip (LOC). The whole system was enclosed in a lead shielded box with a double electronic interlock system put in place to protect operators from being exposed inadvertently to ionizing radiation.

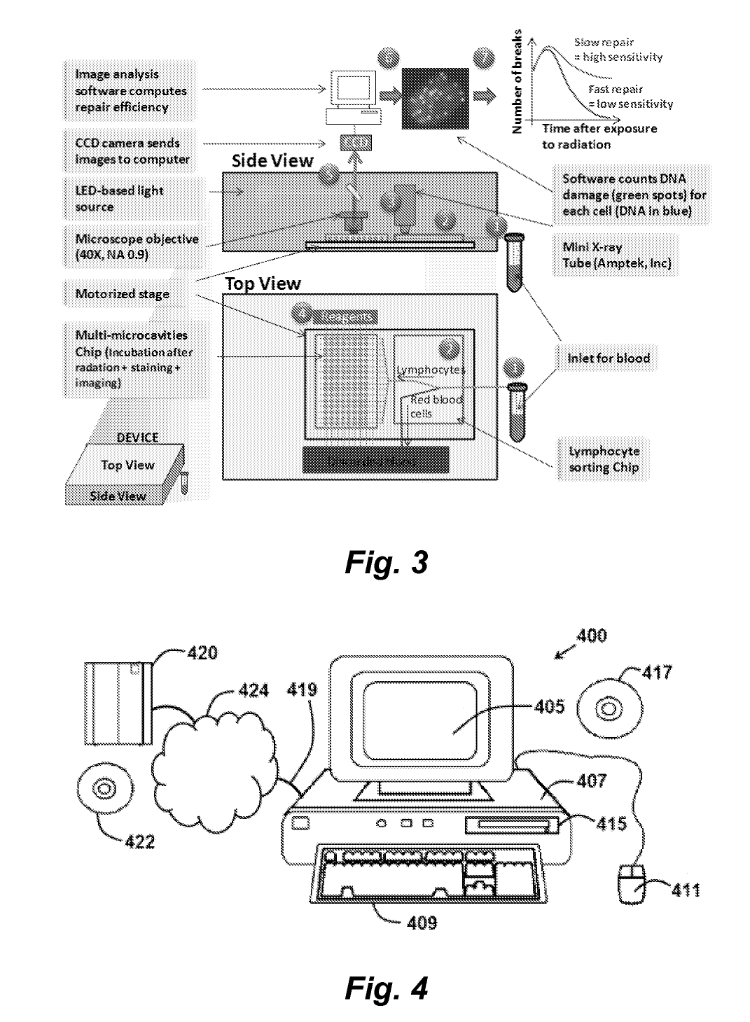

Another illustrative embodiment of this device that is well suited for commercial use is illustrated in FIG. 3. In this design, the microfluidics chip is modified to allow blood as an input instead of flowing human breast cells. This modification incorporates a microfluidics chip able to sort blood cells. In order to be able to induce DNA damage in a blood sample, one needs to use blood cells with a nucleus (leukocytes rather than erythrocytes). Therefore, after being pumped inside a microchannel (e.g., step 1 in FIG. 3), the blood passes through a first LOC designed to isolate lymphocytes and discard red blood cells that have no DNA (e.g., step 2 in FIG. 3).

Various LOC with sorting capacities are known to those of skill in the art (see, e.g. Sethu et al. (2006) Anal. Chem. 78: 5453-5461) and these can readily be integrated with the microfluidic devices described herein. Test cells (e.g., leukocytes flow into a large microchannel at a controllable speed, allowing the MiniX (or other radiation source) to irradiate thousands of cells simultaneously within a few minutes (Step 3 in FIG. 3). The flow rate is set to control the dose given to each cell. In certain embodiments two rates can be used: a slow rate for high dose (e.g., ˜2 Gy) and a fast rate for low doses (e.g., ˜0.1 Gy). High dose repair kinetics can be used to predict acute response to radiation, whereas low dose repair kinetics will be used to predict cancer risk.

The lab-on-a-chip (LOC) illustrated in FIG. 2 can be used to keep the blood cells alive for 4, 8, 12, 16, 20, or 24 hours or more, so that they can repair damage. In certain embodiments 8 time points are used to facilitate the determination of an accurate kinetic constant. Illustrative time points over a 24 hour period are 0.1, 0.5, 1, 2, 4, 8, 16, and 24 hours). It will be recognized, however, that in various embodiments, fewer time points or more time points can be utilized to make the kinetic calculation. Thus, in certain embodiments 2, more preferably at least 3, at least 4, at least 5, at least 6, at least 7, at least 8, at least 9, at least 10, at least 11, at least 12, at least 13, at least 14, at least 15, at least 16, at least 17, at least 18, at least 19, at least 10, at least 21, at least 22, at least 23, or at least 24 time points are used. In certain embodiments, the time points are calculated over about a 3 hour period, or over about a 4 hour period, or over about a 4 hour period, or over about a 5 hour period, or over about a 6 hour period, or over about a 7 hour period, or over about an 8 hour period, or over about a 9 hour period, or over about a 10 hour period, or over about an 11 hour period, or over about a 12 hour period, or over about a 13 hour period, or over about a 14 hour period, or over about a 15 hour period, or over about a 16 hour period, or over about a 17 hour period, or over about an 18 hour period, or over about a 19 hour period, or over about a 20 hour period, or over about a 21 hour period, or over about a 22 hour period, or over about a 23 hour period, or over about a 24 hour period.

In one illustrative embodiment, the microfluidic chip comprises 16 rows of 8 microcavities, allowing to sampling of 4 subjects simultaneously (2 doses in duplicate per subject). This LOC is able to automatically fix the specimen(s) at the appropriate incubation time and label each group of cells with the appropriate reagents (Step 4 in FIG. 3). As the first time points are ready for imaging, a microscope with high numerical aperture objective starts acquiring images for the cavities (microfluidic chamber containing cells) (e.g., ˜500 cells/chamber—step 5 in FIG. 3).

High throughput can also be achieved by dispensing lymphocytes into multi-well plates (e.g., 96 well plates) and imaging is then done via commercial high-content microscope platforms.

Image analysis software (e.g., as described in the Examples) automatically identifies the nuclei and counts the number of RIF per cell for each time point (Step 6 in FIG. 3), allowing the generation of a repair kinetic curve.

In certain embodiments a risk factor for each individual is defined by normalizing to a population or to a subpopulation. For example in certain embodiments the risk factor can be calculated as the ratio of the average or median repair kinetic of for a population to the kinetic measured for the specimen. The faster the repair, the lower that risk factor. This factor can be designated as the “Rad Blood Type” with a value above 1 for persons at risk (see, e.g., step 7 in FIG. 3).

While the foregoing discussion focuses on leukocytes, it will be appreciated that any mammalian cell type can similarly be analyzed as long as the cell contains a nucleus (e.g., nuclear DNA). Thus, in various embodiments, measurements of cells from any tissue or organ are contemplated. In certain embodiments the cells can be from a tumor biopsy in which case the measurement will provide a measure of tumor radiation sensitivity which can be used to inform a therapeutic regimen. For example, if a tumor cell shows low radiation sensitivity while leukocytes show elevated radiation sensitivity, chemotherapy rather than radiation therapy may be indicated.

The configuration of the microfluidic device shown in FIG. 2 is illustrative and not intended to be limiting. Essentially any microfluidic device configured to receive cells, optionally separate those cells, expose the cells to a radiation source, process the exposed cells to label RIFs, and to permit visualization of the RIF can be utilized in the methods and systems described herein. Similarly, the number of microfluidic channels, microfluidic chambers, labeling and reagent channels and chambers, and the like is determined only by the number of samples it is desired to assay, the number of replicates per sample, and the number of time points that are to be assayed for each sample. In certain embodiments the microfluidic devices contemplated herein are configured to permit analysis of at least 2, or at least 4, or at least 6, or at least 8, or at least 10, or at least 12, or at least 14, or at least 16, or at least 18, or at least 20, or at least 30, or at least 40 or at least 50, or at least 100 different samples are contemplated.

In various embodiments essentially any source of ionizing radiation or non-ionizing can be used in the methods and devices described herein as long as the radiation produced is sufficient to induce double strand DNA breaks (DSBs) and produce detectable RIFs.

While in certain embodiments, x-rays are a preferred ionizing radiation, particular as delivered by a mini x-ray tube, the radiation source need not be so limited. Other sources of ionizing radiation are also contemplated. Illustrative sources include, for example, radionuclides (e.g., ⁶⁰Co, ¹⁵³Sm, ¹⁸⁶Re, ¹⁹⁸Au, ¹⁶⁵Dy, ⁹⁰Y, and the like) are also contemplated. Illustrative sources of non-ionizing radiation include for example ultraviolet radiation (UVA and/or UVB).

In various embodiments the use of any of a number of reagents that permit labeling of regions at DNA double stranded breaks is contemplated. Typically such reagents compromise an antibody that specifically binds one or more macromolecules (e.g., proteins) involved in the repair process at the break site attached to a detectable label. In certain embodiments preferred detectable labels include, but are not limited to fluorescent labels (e.g., chemical fluorophore and/or quantum dots). Illustrative suitable antibodies include, but are not limited to antibodies that bind to the phosphorylated form of histone H2AX molecules (γH2AX) (see, e.g., Rogakou et al. (1998) J. Biol. Chem., 273: 5858-5568), and antibodies to P53 binding protein 1 (53BP1).

In certain embodiments the antibody is attached (directly or through a linker) to the label. In other embodiments, a separate labeling reagent (e.g., a labeled anti-IgG antibody) is used to tag and label the DSB specific/localized antibodies.

Any of a number of image analysis systems can be used to capture images of the labeled cells. In various embodiments the image analysis system comprises a microscope objective and a digital camera one or both of which can be under control of a computer. In certain embodiments the image analysis system comprises a single objective and/or detector, while in other embodiments, multiple objective and/or detectors (e.g. digital cameras and/or imaging chips) are utilized permitting simultaneous acquisition of data from a number of samples.

In various embodiments the microfluidic device is mounted on a movable stage to permit the chambers in the microfluidic device to be aligned under the objective(s)/image analysis system. In certain embodiments the microscope objective can be moved to facilitate such alignment.

In various embodiments, the microfluidic devices used in the assay methods and systems described herein can be coupled to (e.g., in fluid communication with) other devices to facilitate sample processing and/or such devices can be incorporated into the microfluidic assay device. Microfluidic devices for sample processing (e.g., isolation of leukocytes from blood) are known to those of skill in the art. For example, in one system described by Sethu et al. (2006) Anal. Chem. 78: 5453-5461, a microfluidic cassette provides three inlets and one outlet. The sample collection (outlet end) has a sample outlet and an inlet buffer (e.g., phosphate-buffered saline (PBS)). The sample loading end has two inlets, for whole blood and one for deionized water. The water is divided into two streams that flank the whole blood stream leading a serpentine lysis channel in which erythrocytes are preferentially lysed by exposure to the water, resulting in enrichment of the sample for leukocytes. This is only one illustrative sample processing cassette that can readily be combined with or incorporated into the devices described herein. Numerous other such sample processing modules will be known to those of skill in the art.

In certain embodiments porous filters can be used to keep lymphocytes inside cavities while clearing debris from lysed erythrocytes.

Any of a number of approaches can be used to convey the fluids, or mixtures of reagents, particles, cells, etc. along the flow paths and/or channels of the devices described herein. Such approaches include, but are not limited to gravity flow, syringe pumps, peristaltic pumps, electrokinetic pumps, bubble-driven pumps, air pressure driven pumps, and the like.

As indicated above, in various embodiments, integrated systems for the exposure of cells to ionizing radiation and the collection and analysis of those exposed cells are contemplated. Such integrated systems can, optionally, further provided for the compilation, storage and access of data and databases pertaining to radiation sensitivity assays. In certain embodiments the integrated systems typically include a digital computer with software including an instruction set for analyzing cells to detect and/or quantify radiation induced foci (RIFs) as described herein. Alternatively, or in addition, the computer can provide for one or more of high-throughput sample control software, image analysis software, collected data interpretation software, a robotic control armature for transferring solutions from a source to a destination operably linked to the digital computer, an input device (e.g., a computer keyboard) for entering subject data to the digital computer, or to control analysis operations or high throughput sample transfer by the robotic control armature. Optionally, the integrated system further comprises valves, concentration gradients, fluidic multiplexors and/or other microfluidic structures for interfacing to a microfluidic device as described.

In various embodiments readily available computational hardware resources using standard operating systems can be employed and modified according to the teachings provided herein, e.g., a PC running as an operating system WIN7®, Unix, Linux, OS10, and the like and/or one or more main frame computers, and/or one or more distributed computational systems using, for example, distributed computational capacity on a local network and/or on the internet.

Current art in software technology is adequate to allow implementation of the methods taught herein on a computer system. Thus, in certain embodiments, the systems can comprise a set of logic instructions (either software, or hardware encoded instructions) for performing one or more of the methods as taught herein. For example, software for providing the data and/or statistical analysis can be constructed by one of skill using a standard programming language such as Unix, Basic Fortran, Java, or the like. Such software can also be constructed utilizing a variety of statistical programming languages, toolkits, or libraries.

FIG. 4 schematically illustrates an information appliance (or digital device) 400 that can be understood as a logical apparatus that can read instructions from media 417 and/or network port 419, that can optionally be connected to server 420 having fixed media 422. Apparatus 400 can thereafter use those instructions to direct server or client logic, as understood in the art, to embody aspects of the analytical methods and/or system operations described herein. One illustrative, but non-limiting, type of logical apparatus that may be so utilized is a computer system as illustrated in 400, containing CPU 407, optional input devices 409 and 411, disk drives 415 and optional monitor 405. Fixed media 417, or fixed media 422 over port 419, may be used to program such a system and may represent a disk-type optical or magnetic media, magnetic tape, solid state dynamic or static memory, etc. In certain embodiments, the methods described herein, especially the analytic methods, may be embodied in whole or in part as software recorded on this fixed media. Communication port 419 may also be used to initially receive instructions that are used to program such a system and may represent any type of communication connection.

Various programming methods and algorithms, including genetic algorithms and neural networks, can be used to perform aspects of the data collection, correlation, and storage functions, as well as other desirable functions, as described herein. In addition, digital or analog systems such as digital or analog computer systems can control a variety of other functions such as the display and/or control of input and output files. Software for performing the electrical analysis methods described herein are also included in the computer systems of the invention.

There are many formats, materials, and size scales for constructing the microfluidic devices described herein and various integrated fluidic systems. In certain embodiments the devices described herein invention (including the microfluidic channels) are made of PDMS (or other polymers) fabricated using a technique called “soft lithography”. PDMS is an attractive material for a variety of reasons including, but not limited to: (i) low cost; (ii) optical transparency; (iii) ease of molding; (iv) elastomeric character; (v) surface chemistry of oxidized PDMS can be controlled using conventional siloxane chemistry; (vi) compatible with cell culture (non-toxic, gas permeable). Soft lithographic rapid prototyping can be employed to fabricate the desired microfluidic channel systems.

One illustrative version of soft lithographic methods involves preparing a master (mold) (e.g., an SU-8 master) to form the microchannel/microchamber system, pouring a pre-polymer onto the master and curing it to form a cured patterned replica (e.g., PDMS polymer replica), removing the replica from the master and trimming and punching tubing inlets as required, optionally exposing the polymer to a plasma (e.g., to an O₂ plasma) and optionally bonding the polymer to a substrate (e.g., a glass substrate).

Another useful property of PDMS and other polymers is that their surface can be chemically modified in order to obtain the interfacial properties of interest (see, e.g., Makamba et al. (2003) Electrophoresis, 24(21): 3607-3619). On illustrative method of covalently functionalizing PDMS is to expose it to an oxygen plasma, whereby surface Si—CH3 groups along the PDMS backbone are transformed into Si—OH groups by the reactive oxygen species in the plasma. These silanol surfaces are easily transformed with alkoxysilanes to yawed many different chemistries (see, e.g., Silicon Compounds: Silanes and Silicones, Gelest, Inc.: Morrisville, Pa., 2004; p. 560; Hermanson et al. (1992) Immobilized affinity ligand techniques, Academic Press, San Diego, Calif. 1992).

In certain embodiments the master mold is typically a micromachined mold. Molds can be patterned by any of a number of methods known to those of skill in the in the electronics and micromachining industry. Such methods include, but are not limited to wet etching, electron-beam vacuum deposition, photolithography, plasma enhanced chemical vapor deposition (PECVD), molecular beam epitaxy, reactive ion etching (RIE), and/or chemically assisted ion beam milling (CAIBM techniques), and the like (see, e.g., (1997) The Handbook of Microlithography, Micromachining, and Microfabrication, Soc. Photo-Optical Instru. Engineer, Bard & Faulkner (1997) Fundamentals of Microfabrication, and the like).

Another illustrative micromachining method uses a high-resolution transparency film as a contact mask for a thick photoresist layer. Multilayer soft lithography improves on this approach by combining soft lithography with the capability to bond multiple patterned layers of elastomer. Basically, after separate curing of the layers, an upper layer is removed from its mold and placed on top of the lower layer, where it forms a hermetic seal. Further curing causes the two layers to irreversibly bond. This process creates a monolithic three-dimensionally patterned structure composed entirely of elastomer. Additional layers are added by simply repeating the process. The ease of producing multilayers makes it possible to have multiple layers of fluidics, a difficult task with conventional micromachining.

In various embodiments, single-layer or multi-layer PDMS devices are contemplated. In illustrative approach, a network of microfluidic channels is designed in a CAD program. This design is converted into a transparency by a high-resolution printer; this transparency is used as a mask in photolithography to create a master in positive relief photoresist. PDMS cast against the master yields a polymeric replica containing a network of channels. The surface of this replica, and that of a flat slab of PDMS, can be oxidized in an oxygen plasma. These oxidized surfaces seal tightly and irreversibly when brought into conformal contact. Oxidized PDMS also seals irreversibly to other materials used in microfluidic systems, such as glass, silicon, silicon oxide, and oxidized polystyrene. Oxidation of the PDMS has the additional advantage that it yields channels whose walls are negatively charged when in contact with neutral and basic aqueous solutions; these channels support electroosmotic pumping and can be filled easily with liquids with high surface energies (especially water).

The fabrication methods described herein are illustrative and not limiting. Using the teachings provided herein, numerous other photolithographic and/or micromachining techniques can be used to fabricate the devices described herein. The micromachining and soft lithography methods described above, as well as many others, are well known to those of skill in the art (see, e.g., Choudhury (1997) The Handbook of Microlithography, Micromachining, and Microfabrication, Soc. Photo-Optical Instru. Engineer, Bard & Faulkner (1997) Fundamentals of Microfabrication; McDonald et al. (2000) Electrophoresis, 21(1): 27-40).

As noted above, in certain embodiments, the methods described herein are preferably implemented using microfluidic devices preferably integrated into a system for performing the determination of radiation sensitivity as described herein. In certain embodiments the microchannels comprising the microfluidic devices have characteristic dimensions ranging from about 100 nanometers to 1 micron up to about 500 microns. In various embodiments the characteristic dimension ranges from about 1, 5, 10, 15, 20, 25, 35, 50 or 100 microns up to about 150, 200, 250, 300, or 400 microns. In some embodiments the characteristic dimension ranges from about 20, 40, or about 50 microns up to about 100, 125, 150, 175, or 200 microns. In various embodiments the wall thickness between adjacent fluid channels ranges from about 0.1 micron to about 50 microns, or about 1 micron to about 50 microns, more typically from about 5 microns to about 40 microns. In certain embodiments the wall thickness between adjacent fluid channels ranges from about 5 microns to about 10, 15, 20, or 25 microns.

In various embodiments the depth of a fluid channel ranges from 5, 10, 15, microns to about 1 mm, 800 microns, 600 microns, 500 microns, 400 microns, 300 microns, 200 microns, 150 microns, 100 microns, 80 microns, 70 microns, 60 microns, 50 microns, 40 microns, or about 30 microns. In certain embodiments the depth of a fluid channel ranges from about 10 microns to about 60 microns, more preferably from about 20 microns to about 40 or 50 microns. In some embodiments the fluid channels can be open; in other embodiments the fluid channels may be covered.

While the foregoing discussion focuses on evaluation of radiation-induced foci (RIF), in various embodiments, the methods contemplated herein need not be so limited. For example, when performing RIF assay on lymphocytes from human peripheral blood, spontaneous damage is observed in non-irradiated controls, with an average baseline between 0 to 1 foci per cell. Moreover, it was a surprising discovery that baseline foci levels correlate with radiation sensitivity in animals (e.g., Balb/C foci background levels are higher than CB57 mice). Thus, it is believed that baseline foci levels provide another surrogate marker of radiation sensitivity. Moreover, such baseline foci-levels can readily be evaluated, e.g., in a small drop of blood using finger prick devices.

Baseline foci-levels provides a somewhat less robust assay for radiation sensitivity as elevated levels of DNA breaks may not only reflect genetic defects in DNA repair, but are also a function of other factors such as antioxidant-poor diets, elevated stress, environmental factors, and the like. Nevertheless this simplified assay finds utility in a number of contexts. For example, monitoring of baseline foci-levels provides a mechanism to monitor the impact of diet, life style changes, environmental damage, and the like on a subject. Monthly monitoring can help identify successful approaches to lower or control daily DNA damage in an individual and the right partnership with nutritionists, diet and sport industries can lead to improve personal health.

The measurement of baseline foci-levels can also be used to evaluate exposure to radiation workers (e.g. medical imaging, nuclear industry, airline industry, military), and the like.

Human blood contains 1-2000 PBMCs per microliter. DNA damage and repair processes can be monitored in a relatively small number of nucleated leukocytes as described above. The isolation of such cells from small volumes (<1 milliliter) of blood is valuable for use in “at-home” cell-health monitoring protocols.

In various embodiments blood collection can be at home, e.g., via a lancet device, with for example, a plastic capillary for collection and dispensing into a tube/receptacle with fixative reagent (PFA), anti-coagulant (EDTA) and lysis buffer (to remove erythrocytes). In certain embodiments the lancet can be integrated into the reagent/capillary device and optionally an analysis device. Simple microfluidics can be used to perform immunocytochemistry of DNA repair markers.

One illustrative, but non-limiting single use lancet and capillary loading mechanism is described by Zimmerman (2011) Single use lancet and capillary loading mechanism for complete blood count point of care device, MIT Master's Dissertation. This dissertation describes a single use lancet device connected to a blood collection device that holds a blood collection capillary, a reagent capillary, and a chamber for liquid waste. Together the lancet and blood collection device make the “consumable” part of the device that is used only once per sample and then disposed of. A final module is a consumable loader. This is a component of the blood device that can be used to analyze blood samples for a single for many collection events. Other illustrative, but non-limiting lancet and blood collection/analysis devices are described in U.S. Patent Publication Nos: US 2012/0157881 A1, US 2010/0100113 A1, US 2007/0265654 A1, US 2005/0145520 A1, US 2005/0131441 A1, and the like.

One illustrative, but non-limiting methodology for isolation of intact nucleated leukocytes and other blood components for the monitoring of blood-based health markers including but not limited to: DNA damage in cells, lipid variations in blood and serum, cancer biomarkers, small molecule markers of health and fitness and detection of radiation exposures can be performed as follow. Blood collection can be done on site (e.g. at home) via a small kit including all necessary reagents and devices. In certain embodiments the kit includes an alcohol swab for site sterilization, a lancet device, a capillary for blood collection, red blood cell lysis and fixative reagents in the form of small volumes of liquid in dedicated sealable tubes or provided in an integrated module. Blood can be collected via a finger prick with the lancet device at a site sterilized with the alcohol swab. The supplied plastic capillary (and/or integrated collection device_is for collection of approximately 50-100 microliters of whole blood which can be dispensed into a tube or chamber with anti-coagulant (e.g., EDTA/Citric acid), fixative reagent (e.g., paraformaldehyde or gluteraldehyde), and lysis buffer (to disrupt erythrocytes).

Whole blood is acquired and immediately transferred and mixed with fixative/lysis reagent mix. In various embodiments fixative and red blood cell lysis mixtures may include paraformaldehyde, gluteraldehyde, citric acid, EDTA, ammonium chloride, buffers, among other reagents useful in retaining PBMC integrity and disruption of red blood cells. The preparation of blood cells in this manner allows for simpler microfluidics to be used to perform immunocytochemistry of DNA repair markers. Additional advantages include: at-home sampling, shipping of samples to central process location, immediate trapping of cell status at time of blood draw, eliminates the need for phlebotomy.

Foci determination can be performed, e.g., using a microfluidic system (e.g., LOC) as described above.

The foregoing description and referenced Figures are intended to be illustrative and not limiting. Using the teachings provided herein other device/system configurations, and variations of the methods will be available to one of skill in the art.

### EXAMPLES

The following examples are offered to illustrate, but not to limit the claimed invention.

### Example 1

### Evidence for Formation of DNA Repair Centers and Dose-Response Nonlinearity in Human Cells

The concept of DNA “repair centers” and the meaning of radiation induced foci (RIF) in human cells have remained controversial. RIFs are characterized by the local recruitment of DNA damage sensing proteins such as p53 binding protein (53BP1). Here, we provide strong evidence for the existence of repair centers. We used live imaging and mathematical fitting of RIF kinetics to show that RIF induction rate increases with increasing radiation dose, whereas the rate at which RIFs disappear decreases. We show that multiple DNA double-strand breaks (DSBs) 1 to 2 μm apart can rapidly cluster into repair centers. Correcting mathematically for the dose dependence of induction/resolution rates, we observe an absolute RIF yield that is surprisingly much smaller at higher doses: 15 RIF/Gy after 2 Gy exposure compared to approximately 64 RIF/Gy after 0.1 Gy. Cumulative RIF counts from time lapse of 53BP1-GFP in human breast cells confirmed these results. The standard model currently in use applies a linear scale, extrapolating cancer risk from high doses to low doses of ionizing radiation. However, our discovery of DSB clustering over such large distances casts considerable doubts on the general assumption that risk to ionizing radiation is proportional to dose, and instead provides a mechanism that could more accurately address risk dose dependency of ionizing radiation.

DNA damage-sensing proteins localize at sites of DNA double-strand breaks (DSBs) within seconds to minutes following ionizing radiation (IR) exposure, resulting in the formation of immunofluorescently stainable nuclear domains referred to as radiation-induced foci (RIF) (Costes et al. (2006) Radiat. Res. 165: 505-515; Costes et al. (2007) PLoS Comput. Biol. 3: e155; Rogakou et al. (1998) J. Biol. Chem. 273: 5858-5868). RIF numbers are routinely used to assess the amount of DNA damage and repair kinetics after different treatments (Costes et al. (2010) Mutat. Res. 704: 78-87). However, there is a controversy surrounding the question of whether there is a 1:1 correspondence between RIF and DSBs. For example, pulse field gel electrophoresis (PFGE) analysis suggests that DSBs decay exponentially with time immediately after exposure (Stenerlow et al. (2003) Radiat. Res. 159: 502-510). In contrast, DNA damage-sensing proteins do not instantaneously detect DSBs, leading to delayed kinetics for both detection and resolution. More specifically, the maximum number of 53BP1 or γH2AX RIF is not reached until 15 to 30 min after exposure, and the yield of DSBs predicted by RIF is typically lower than the expected 25-40 DSB/Gy measured by PFGE (Costes et al. (2010) Mutat. Res. 704: 78-87).

Dose response provides another assay for assessing the relationship between DSBs and RIF. Based on theoretical Monte Carlo simulations and PFGE measurements (Goodhead and Nikjoo (1989) Int. J Radiat. Biol. 55: 513-529; Erixon and Cedervall (1995) Radiat. Res. 142: 153-162), the frequency of DSBs should be highly correlated with radiation dose. Confirming this prediction, two research groups reported that RIF number is proportional to radiation dosage from 1 mGy to 2 Gy (Rothkamm and Lobrich (2003) Proc. Natl. Acad. Sci. USA, 100: 5057-5062; Asaithamby and Chen (2009) Nucleic Acids Res. 37: 3912-3923). In both studies, methods were applied to identify “real” RIF at low doses, where frequencies may be close to background levels before IR (e.g., 10 mGy would lead to about 0.3 DSB/cell). They either used cells with very low γH2AX background foci (i.e., 0.05 background foci/cell in primary human lung MRC-5 fibroblasts) (Rothkamm and Lobrich (2003) Proc. Natl. Acad. Sci. USA, 100: 5057-5062), or performed live studies with a tagged DNA damage marker (i.e., 53BP1-GFP) and disregarded foci that were present before exposure to IR (Asaithamby and Chen (2009) Nucleic Acids Res. 37: 3912-3923). However, there were discrepancies between these two studies. One study reported a 1:1 correspondence between RIF and DSBs, with a maximum of 35 γH2AX RIF/Gy at 3 min post-IR exposure (Rothkamm and Lobrich (2003) Proc. Natl. Acad. Sci. USA, 100: 5057-5062), whereas the other study reported RIF frequencies were maximal much later (i.e., 30 to 60 min post-IR), with different proportionality; i.e., 16-20 53BP1-GFP RIF/Gy for human HT1080 and 60 53BP1-GFP RIF/Gy for immortalized human bronchial epithelial cells (Asaithamby and Chen (2009) Nucleic Acids Res. 37: 3912-3923). These discrepancies cast some doubts on the one-to-one correspondence between RIF and DSB and also show that cell type and methods of analysis both play a crucial role in RIF quantification. Furthermore, dose/response linearity is not always observed. For example, in normal human fibroblasts (Costes et al. (2006) Radiat. Res. 165: 505-515) and in hamster V79 cells (MacPhail et al. (2003) Int. J. Radiat. Biol. 79: 351-358), we observed a maximum of 18-24 γH2AX RIF/Gy after exposure to less than 1 Gy of X-rays, compared to 13-15 γH2AX RIF/Gy for 1-4 Gy. Similarly, human fibroblasts showed a slight decrease with averages ranging from 21 to 17 RIF/Gy between 0.05 and 0.25 Gy, which was consistent across 18 independent lines (Wilson et al. (2010) Mutat. Res. 683: 91-97).

Most studies measure RIF only at discrete times after the induction of damage. This means that the temporal complexity of the biochemical response, primarily initiated by DNA damage, is often neglected. However, temporal delays in RIF formation relative to DSBs as well as different resolution times for RIF complicate the interpretation of RIF numbers. In addition, even when kinetic studies are performed, the number of RIF reported at any given time after IR can never reflect the total number of RIF that have been produced by IR, as all RIF that have already been resolved or that have not yet been produced are not counted.

Here, we present a mathematical formalism that extracts the absolute number of RIF from RIF kinetics data. By integrating this biophysical model with a standardized high-content imaging methodology (Costes et al. (2007) PLoS Comput. Biol. 3: e155), we demonstrate the ability to get reproducible RIF results from different research laboratories. Miniaturization of cell cultures, using microwell slide technology, were also applied to further accelerate and normalize sample treatment and processing. This comprehensive quantitative analysis challenges the concept of linearity between IR dose and RIF yield and suggests the existence of DNA repair centers in human cells.

### Results

Validation of RIF Yield and Formation-Disappearance Kinetics Models Using Live Cells Exposed to X-Rays.

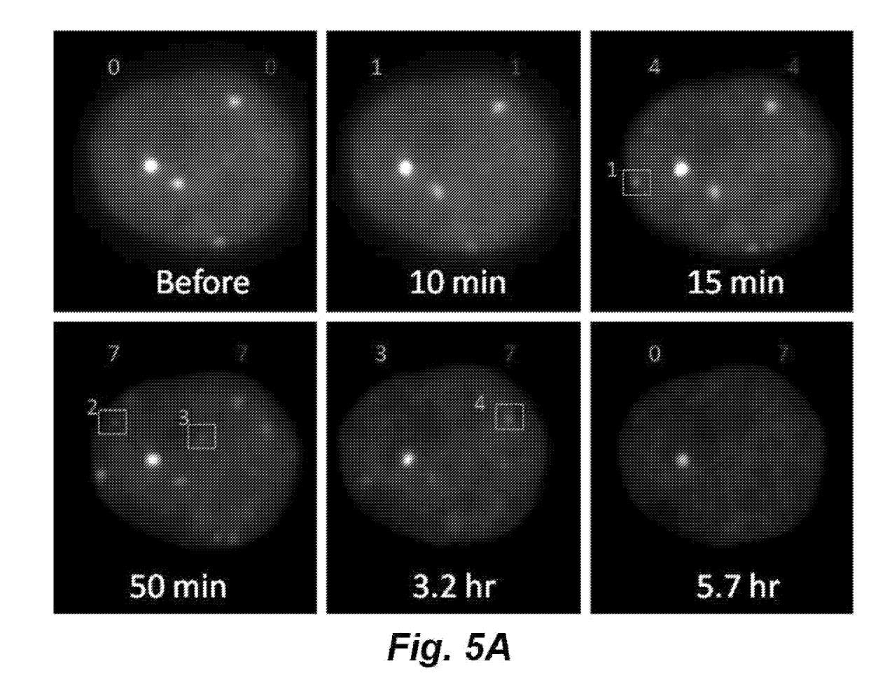

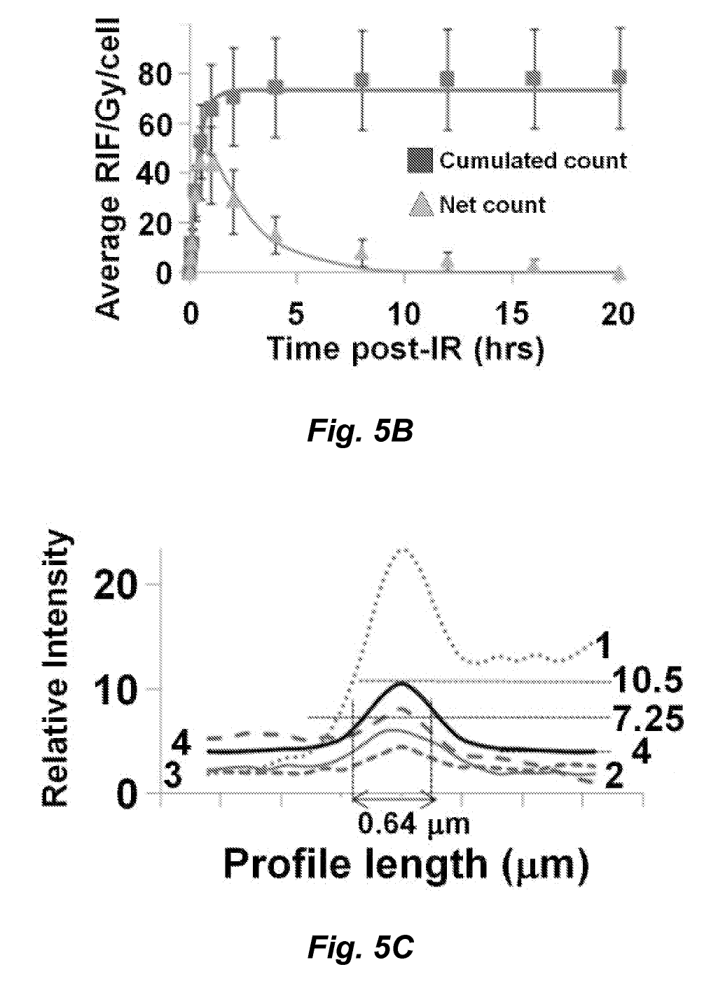

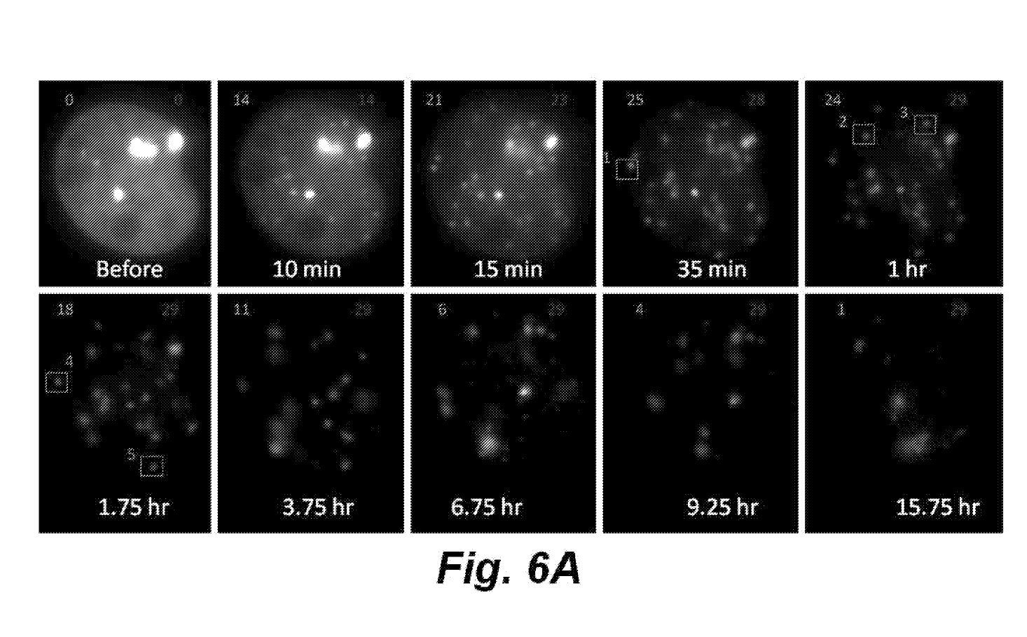

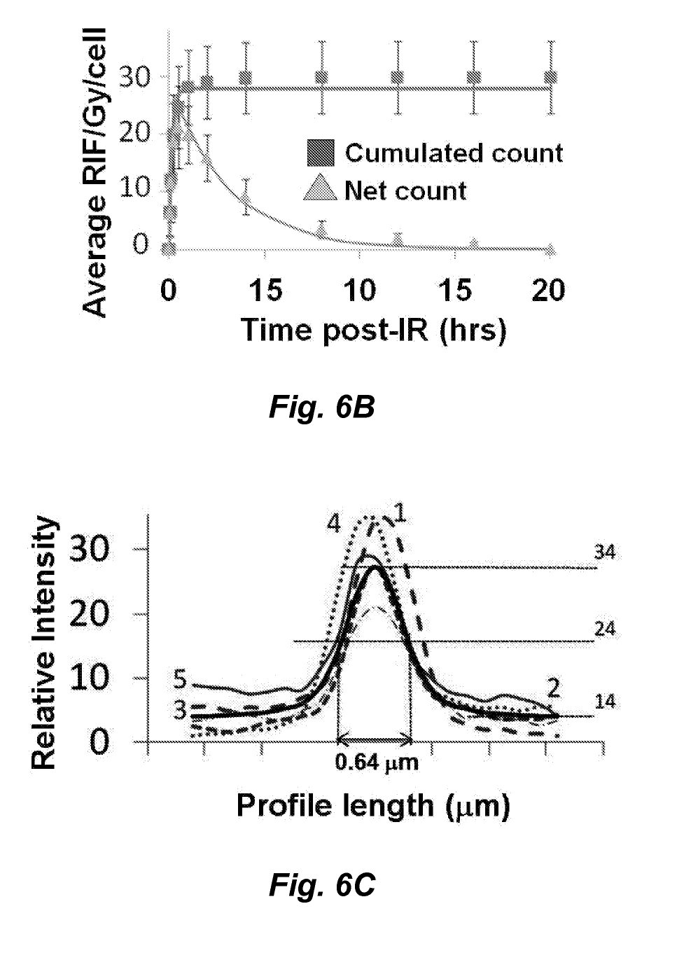

We propose a mathematical model to fit the kinetics of RIF formation, which can deduce the absolute number of RIF produced by a given dose of IR from the net number of RIF measured at any time point (see Materials and Methods). Live cell imaging is ideal to validate such a model, because it simultaneously measures the number of RIF at any given time and the number of RIF accumulated since the time of exposure to X-rays. To test the validity of our model, we fitted with Eq. 1 the number of RIF measured in MCF10A transiently transfected with 53BP1-GFP. Both the number of RIF counted at each time frame, as well as the cumulative number of RIF counted after IR exposure, were scored (representative snapshots and kinetics are shown in FIGS. 5A and 2A for 0.1 and 1 Gy, respectively). If Eqs. 1 and 2 were correct, the cumulative RIF counts (top curves shown in FIGS. 5B and 6B) should converge over time to a constant value equal to the total number of RIF/Gy (α).

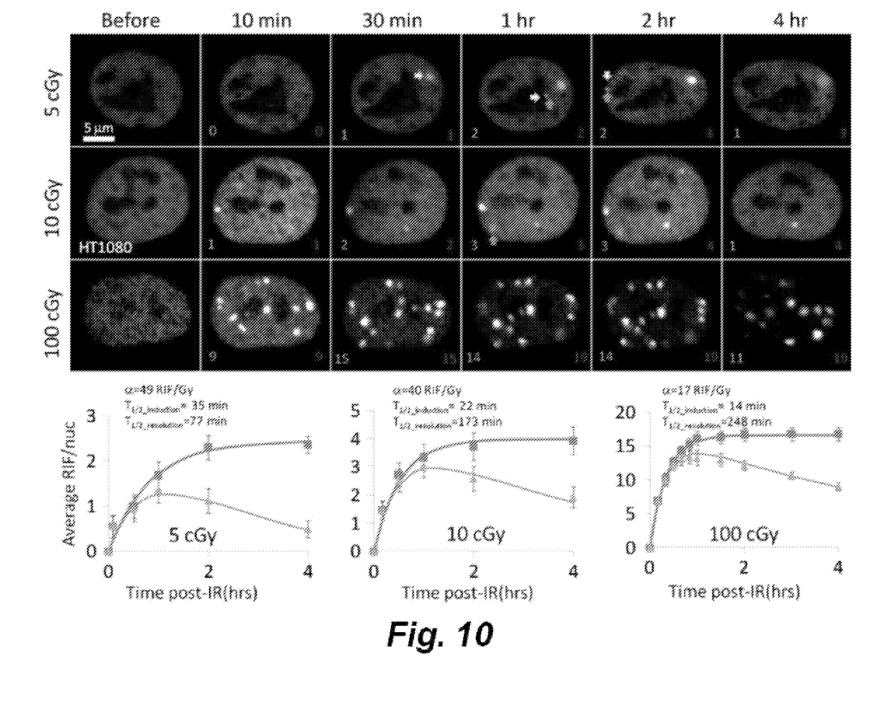

Confirming this biophysical model, fits of the net kinetics (bottom curves in FIGS. 5B and 6B) with Eq. 1 led to an α value that matched the total cumulated yield. In addition, live cell imaging revealed that the total number of RIF produced by IR was not proportional to dose, and was relatively lower at higher doses (73 RIF/Gy vs 28 RIF/Gy at 0.1 and 1 Gy, respectively). In addition, RIF induced by low doses appeared more slowly and were resolved faster than after 1 Gy, as indicated by the reported formation and resolution half-lives on the graph (T_{1/2}). Three dimensional time lapse using confocal microscopy on human fibrosarcoma HT1080 stably transfected with 53BP1-GFP showed very similar properties for 0.05, 0.1, and 1 Gy (FIG. 10). Finally, monitoring the intensity profiles of individual RIF during time lapse imaging identified changes in RIF size and intensity during focus formation (blue dashed rectangle in FIGS. 5, panel A and 6, panel A). The relative intensity profiles for individual foci (1D intensity cross-section of focus location normalized to the average 53BP1 intensity outside foci regions) and their averages are shown in FIGS. 5, panel C and 6, panel C. Even though no difference in size could be observed, with an average RIF diameter of 0.64 μm for both high and low dose, a threefold increase of RIF intensity was measured after high dose.

High-Content Analysis on Fixed Specimens Confirm Nonlinear RIF Yield with Dose.

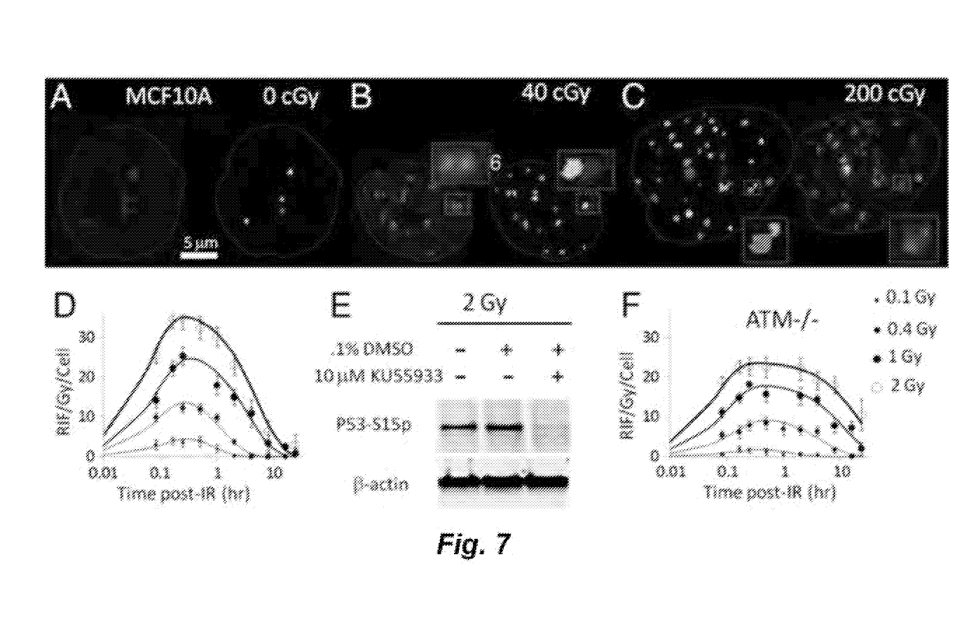

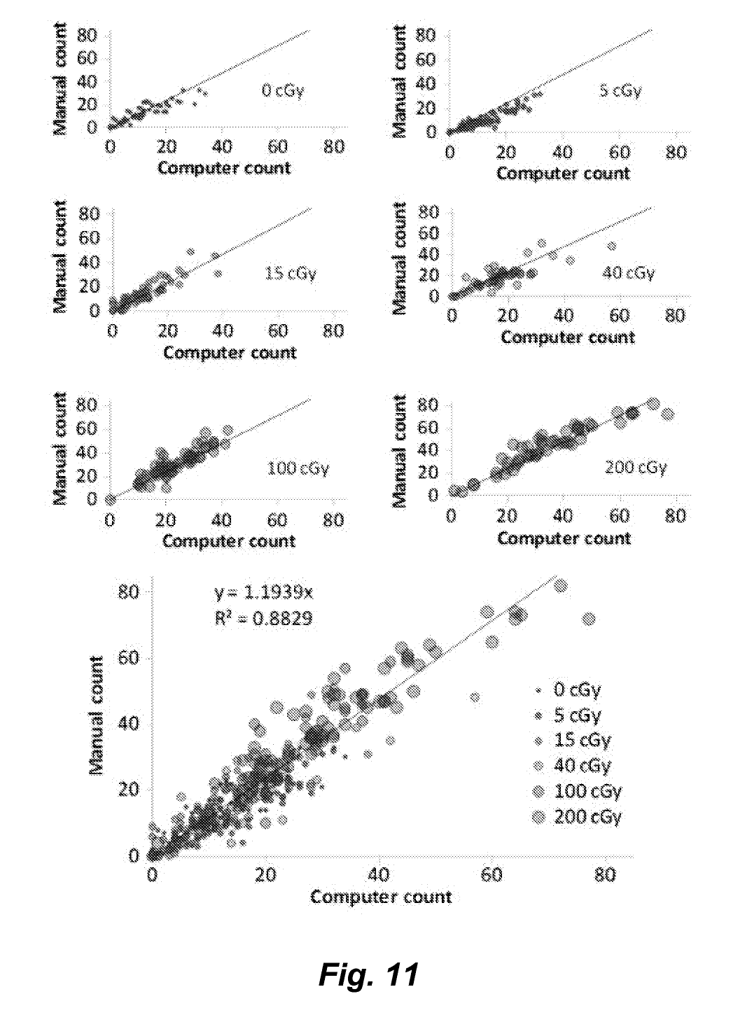

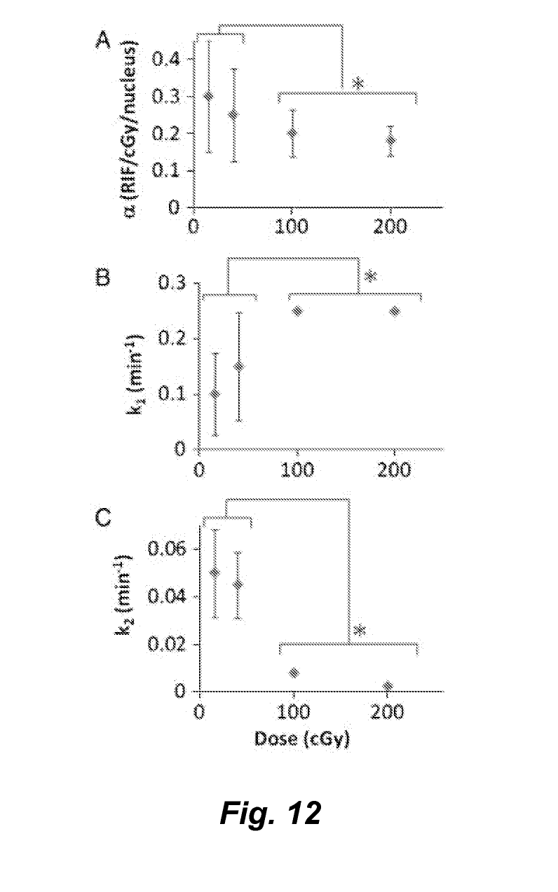

In order to quantify a larger dataset representing endogenous levels of proteins, we analyzed arrays of fixed MCF10A by immunostaining for 53BP1. As described in Materials and Methods, detection of RIF was done automatically, using improved in-house RIF detection algorithms (Costes et al. (2007) PLoS Comput. Biol. 3: e155). The computer scoring obtained in this manner was corroborated for a subset of cells counted manually at 30 min after different doses of X-ray (from 0.05 to 2 Gy; FIG. 11). FIG. 7, panels A-C show representative images for selected doses, showing the efficiency of the algorithm for separating touching foci. Applying this approach for fitting average counts of seven independent experiments measured at various doses of X-rays collected over a 24-h time course, we observed excellent agreement with Eq. 1 (FIG. 7, panel D). All fitted coefficients for Eq. 1 are summarized in Table 1. Similarly to what we observed with 3D time lapse, the absolute number of 53BP1 RIF normalized to dose (a in RIF/Gy per nucleus) decreased approximately 4-fold between 0.1 and 2 Gy (approximately 64±6 to 16±2 RIF/Gy, after 0.1 and 2 Gy, respectively). This decreasing trend was statistically significant (P value<0.01 using t test). RIF kinetics were also dose dependent: RIF formation was twice as fast and RIF resolution was approximately 5 times slower at 2 Gy versus 0.1 Gy (see Table 1). Both k1 and k2 dose dependence were significant (P value<0.05 using one-way ANOVA). To test if the dose-dependent DNA damage response was specific to breast epithelial cells, the same measurements were made on immortalized human skin fibroblasts (HCA2) grown as confluent populations (Costes et al. (2006) Radiat. Res. 165: 505-515), where we observed a similar trend, with a 1.7-fold decrease of RIF yield α, a 2.5-fold increase in 53BP1 RIF induction rate, and a 20-fold decrease in RIF resolution rate between 0.1 and 2 Gy (FIG. 7).

TABLE 1
Fitted parameters for various doses of X-rays, and for delta rays and
track core time response to 1 Gy of 1 GeV/atomic mass unit Fe.

| | Average | | | Standard error | | |
|---|---|---|---|---|---|---|
| | α, | T1/2_1, | T1/2_2 | α, | T1/2_1 | T1/2_2 |
| Dose (Gy) | RIF/Gy | (min) | (h) | RIF/Gy | (min) | (h) |

Controls MCF10A (N = 7)

| | | | | | | |
|---|---|---|---|---|---|---|
| 0.1 | 64 | 5.6 | 1.4 | 6 | 1.3 | 0.5 |
| 0.4 | 38 | 3.4 | 2.0 | 2 | 0.4 | 0.6 |
| 1 | 23 | 2.8 | 3.8 | 3 | 0.5 | 0.6 |
| 2 | 15 | 2.4 | 5.7 | 2 | 0.1 | 1.6 |

ATM inhibition MCF10A (one experiment)

| | | | | | | |
|---|---|---|---|---|---|---|
| 0.1 | 25 | 8.3 | 0.7 | 17 | 5.8 | 0.5 |
| 0.4 | 25 | 5.9 | 3.5 | 4 | 1.1 | 0.6 |
| 1 | 19 | 4.2 | 8.7 | 2 | 0.5 | 1.1 |
| 2 | 12 | 2.6 | 15.4 | 2 | 0.3 | 1.9 |

Live 53BP1-GFP in MCF10A (N = 3, 5 to 10 cells per experiment)

| | | | | | | |
|---|---|---|---|---|---|---|
| 0.1 | 73 | 15.4 | 1.4 | 5 | 1.6 | 0.2 |
| 1 | 28 | 6.5 | 2.1 | 3 | 2.1 | 0.1 |

1 GeV/atomic mass unit Fe in MCF10A (N = 5)

| | | | | | | |
|---|---|---|---|---|---|---|
| 0.17 (delta | 43 | 2.8 | 3.3 | 9 | 0.2 | 0.8 |
| rays) * | | | | | | |
| 27 (core)* | — | 0.1 | 9.6 | — | 0.01 | 1.6 |

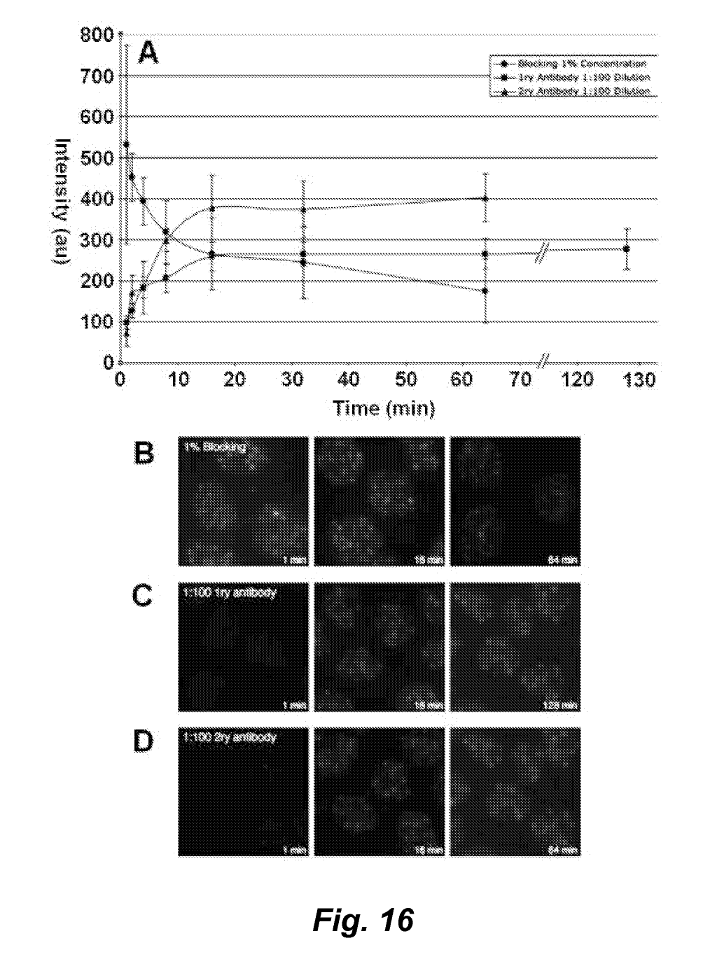

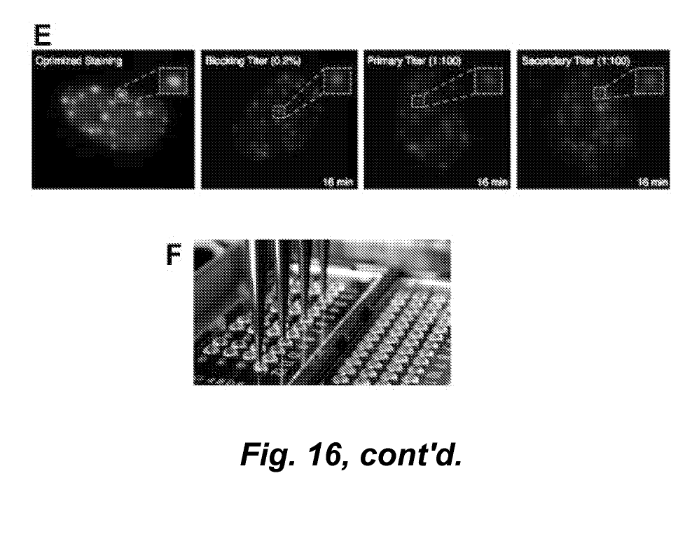

*Dose estimation based on microdosimetry computations of 1 GeV/atomic mass unit Fe exposure (Fig. 16).

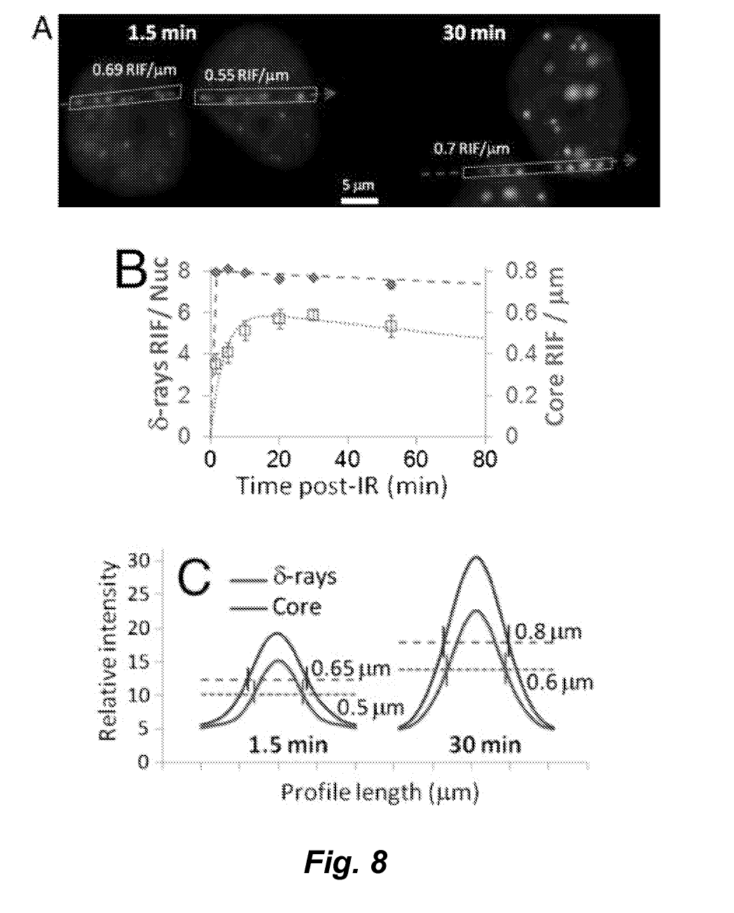

To test the validity of the mathematical model further, we perturbed the rates of RIF formation or RIF removal by inhibiting ataxia telangiectasia mutated (ATM) kinase activity with KU55933 (see Materials and Methods). ATM inhibition was confirmed by measuring the reduction of phosphorylated p53 at S15 (FIG. 7, panel E). As expected, the overall number of RIF was largely diminished (FIG. 7, panel F). However, the same behavior was observed; i.e., RIF yield dropped by 2-fold between 0.1 and 2 Gy (25±17 vs. 12±2 RIF/Gy). Fitted parameters are shown in FIG. 8, panels A-C. Interestingly, detection half-lives were comparable with or without ATM inhibition across all doses, whereas resolution was significantly slower at high doses when ATM was inhibited (significant difference between 15.4±1.8 h with inhibition and 5.7±1.6 h without inhibition, after 2 Gy). This indicates that DSBs requiring longer repair time are still being detected at the same rate, in the absence of ATM kinase activity.

RIF Analysis in Human Cells Exposed to Dense IR Reveals Self-Exclusion of Nearby RIF.

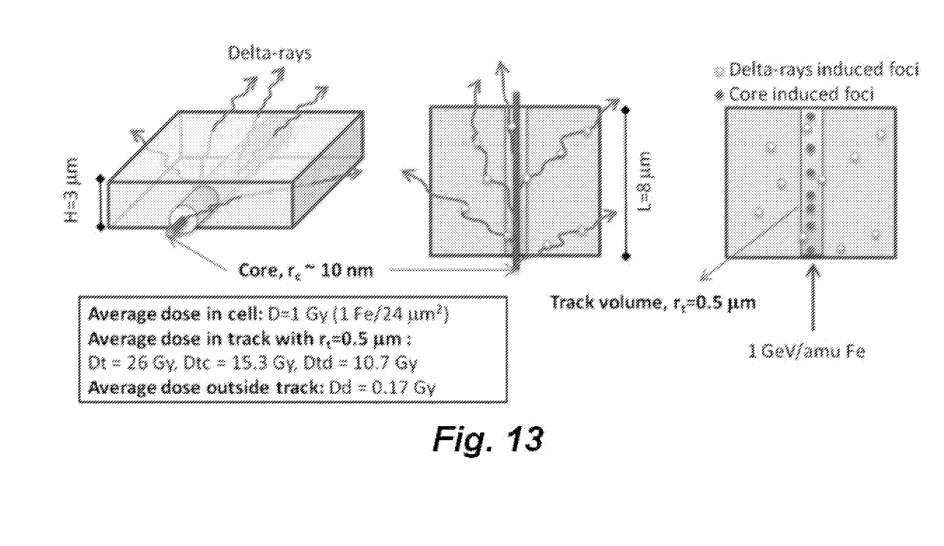

In order to further explore the saturation effect of RIF numbers observed at higher dose, one would need to look at the DNA damage response for doses of X-rays higher than 2 Gy. However, at such high doses there are several confounding factors: (i) there is the difficulty of resolving high numbers of RIF in the nucleus, and (ii) the physiological effects on the cells manifest at higher doses (e.g., toxicity, cell cycle arrest, etc.). In order to circumvent these issues, we used high-energy Fe ions (1 GeV/atomic mass unit), referred to as HZE (High Z and energy). As illustrated in FIG. 13, HZE particles typically deposit part of their energy along linear tracks referred to as cores, and the other part is deposited from electrons randomly outside the core (i.e., delta rays). The radius of the core is about 10 nm for 1 GeV/atomic mass unit Fe ions, whereas delta rays radiate approximately 270 μm from the track (Costes et al. (2000) Radiat. Res. 154: 389-397; Magee and Chatterjee (1980) J. Phys. Chem. 84: 3529-3536). As we described previously (Costes et al. (2007) PLoS Comput. Biol. 3: e155), we have developed imaging tools that automatically identify these tracks and can discriminate RIF along the tracks from random RIF in the nucleus (presumably generated by delta rays; FIG. 13). In order to account for RIF and physiological chromatin movement over time, all RIF detected within a 0.5-μm radial distance from the particle trajectory were considered “core RIF.” Assuming a radial dose distribution decreasing as the distance square from the core (Chatterjee et al. (1973) Radiat. Res. 54: 479-494; Tobias et al. (1971) Science. 174: 1131-1134), we estimated a dose of 26 Gy within the 0.5-μm radius track, and 0.17 Gy from delta rays dispersed outside that region (FIG. 13). Thus, HZE particle radiation allowed us to compare two compartmentally distinct radiation doses within the same cell (representative images shown in FIG. 8, panels A and B). We noted that core RIF sizes and intensities (FIG. 8, panel C) were comparable to 1-Gy X-ray foci (FIG. 6, panel C) as early as 1.5 min post-IR. However, core RIF were larger and brighter by 30 min post-IR. In contrast, delta-ray RIF size and intensity kinetic was comparable to X-rays (FIG. 8, panel C vs FIG. 5, panel C, respectively).

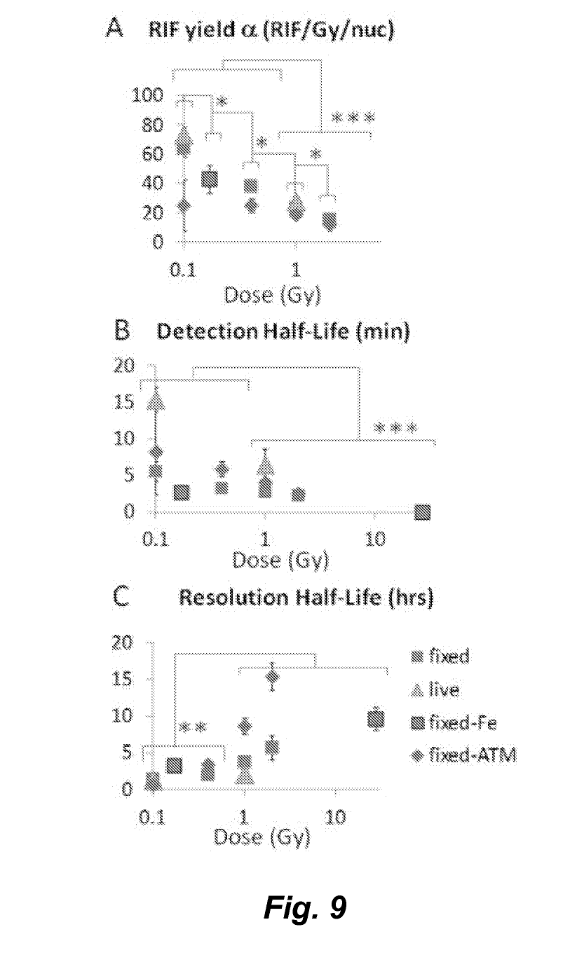

In addition, our results confirmed what was observed for X-rays; i.e., high local doses along the track led to much faster RIF induction (approximately 5-s half-lives) and slower RIF resolution (approximately 10-h half-lives) than in the low-dose region of the delta rays (2.8 min and 3.3 h, respectively). The fitted coefficients are plotted against all other conditions studied in this work in FIG. 9, panels A-C and listed in Table 1. Note that the measured RIF yield along the tracks was fitted to be 0.83 RIF/μm but could not be plotted against other a values in FIG. 9, panel A because it was in a different unit.

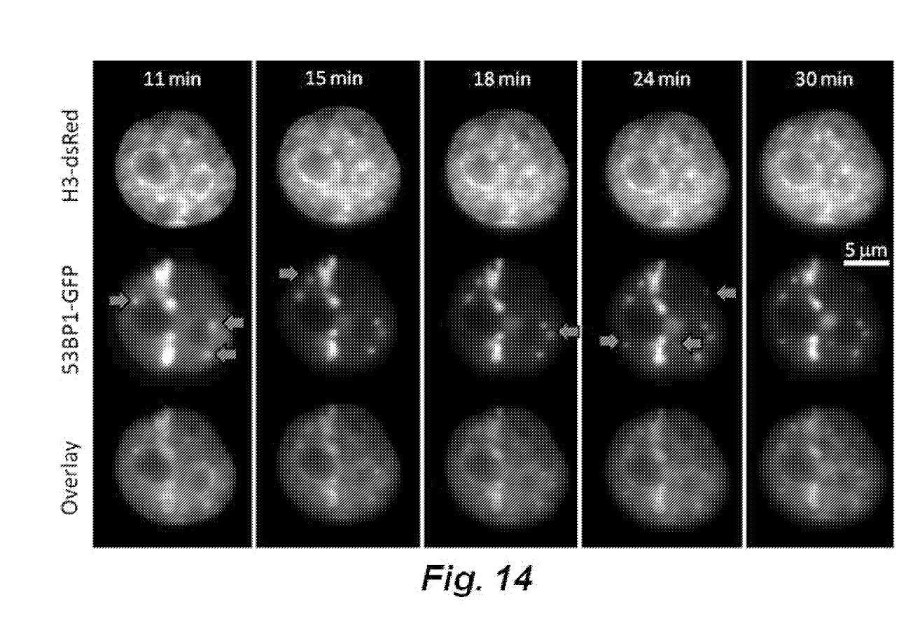

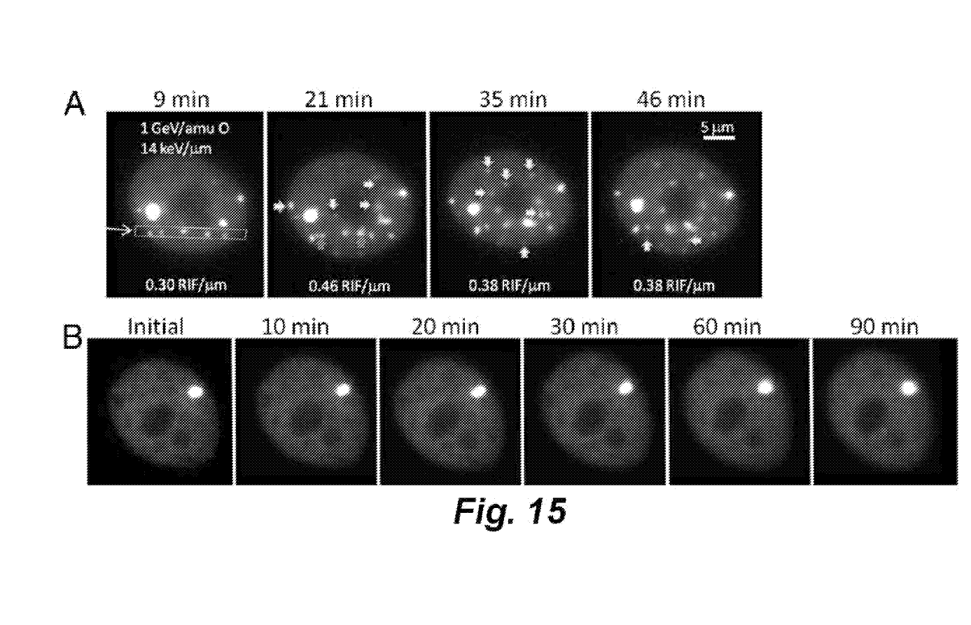

Similar differences in RIF kinetics between track RIF and delta-ray RIF were also observed in live cell imaging of MCF10A cells transiently transfected with 53BP1-GFP (FIG. 14). Time-lapse imaging showed that after initial foci formation there were few new foci appearing along the tracks, whereas new delta-ray RIF outside the track kept appearing during the initial 30-min post-IR period. Similar results were observed also in stably transfected human bronchial epithelial cells exposed to 1 GeV/atomic mass unit 0 ions (FIG. 15).

### Discussion

Single time or single dose measurements are snapshots and might not capture the complexity of the IR response of DNA damage sensing proteins. Here, we present a methodology and a mathematical kinetic model that can characterize the DNA damage response simultaneously across both time and dose levels. Our results provide a more accurate model of RIF dose response, and underscore fundamental concerns about static image data analysis in the dynamic environment of the living cell. We observe that as the number of DSB increases in a cell, the number of RIF does not increase proportionally and the kinetics of RIF formation/disappearance is altered; RIF appear faster but remain longer in the cells as dose levels increase. These nonlinear processes cast considerable doubts on the general assumption that risk to IR is proportional to dose and could be interpreted as the consequence of DNA repair centers in human cells.

Clustering of DSB into Repair Centers at High Dose.

As recently reviewed (Costes et al. (2010) Mutat. Res. 704: 78-87), most studies in the literature report RIF yield well below the expected 25-40 DSB/Gy measured by PFGE in cells in the G1 part of the cell cycle (Stenerlow et al. (2003) Radiat. Res. 159: 502-510; Rothkamm and Lobrich (2003) Proc. Natl. Acad. Sci. USA, 100: 5057-5062). This probably reflects the fact that what is measured at any time point is the net number of RIF that have formed since radiation, which does not account for RIF that have already been resolved, or for RIF that have not yet appeared. The time-lapse imaging presented here shows clearly that RIF formation continues to occur well beyond initial IR exposure time. In addition, our biophysical model fits well the kinetics curves observed for the number of RIF per nucleus and accounts for these missing RIF. These fits suggest that the absolute RIF yield normalized to dose (a) is not constant but drops 4-fold between 0.1 and 2 Gy. The lower yield of α at high dose cannot be explained by depletion of the pool of 53BP1. Indeed, protein depletion would only lead to dimmer foci, not fewer foci. Furthermore, RIF number saturation cannot be due to overlapping foci because the expected spatial random distribution of DSBs simulated by computer (see Materials and Methods) predicts average distances easily resolvable by light microscopy at the highest dose considered (2 Gy). Similarly, using radiation that deposits a high amount of energy along a tightly defined track, we observe approximately 0.7-0.8 RIF/μm along 1 GeV/atomic mass unit Fe (linear energy transfer, LET=150 keV/μm), contrary to a theoretical value based on physical considerations of approximately 1.1 DSB/μm (Costes et al. (2007) PLoS Comput. Biol. 3: e155). In addition, when cells are exposed to ions with a hundred times higher energy densities (e.g., uranium ions with LET of 14; 300 keV/μm and expected approximately 100 DSB/μm), RIF frequencies remain in the same order of magnitude (i.e., 0.96 XRCC1 RIF/μm) (Jakob et al. (2009) Radiat. Res. 171: 405-418), suggesting full saturation of the number of RIF. One potential explanation for this apparent saturation is the existence of repair centers with a minimum interdistance of approximately 1 μm. If repair centers exist, as the local dose increases, the probability of having two DSBs migrating into one common RIF increases, leading to lower RIF counts per dose, faster induction, and slower resolution.

Note that a distance of 1-2 μm is in good agreement with previous estimate of the distance between two separate DSBs which can explain DSB mis-rejoining data leading to the classic supralinear dose dependence observed for radiation-induced chromosomal rearrangements (Sachs et al. (1997) Int. J. Radiat. Biol. 71: 1-19; Sachs et al. (1999) Math. Biosci. 159: 165-187). Time-lapse imaging also suggests that if DSB clustering takes place, it happens before an RIF is formed, because RIF clustering was not observed within the first 30 min post-IR. On the other hand, we did observe the merging of RIF over hours post-IR. RIF merging over long time course has already been described along high energy density tracks (Aten et al. (2004) Science, 303: 92-95), and has been interpreted as transient clusters that eventually separate again (Jakob et al. (2009) Proc. Natl. Acad. Sci. USA, 106: 3172-3177).

In this work, we hypothesize that DSB clustering occurs rapidly after IR and that RIF formation reflects the repair machinery put in place around one cluster of DSBs. DSB clustering can then be rewritten as follows:

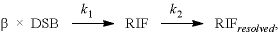

where β(D) is the average number of DSB within one RIF. Assuming 35 DSB/Gy, β=35/α and based on our data, it increases with dose: β˜1 DSB/RIF at 0.4 Gy, suggesting a one-to-one correspondence, whereas there would be β˜2.3 DSB/RIF after 2 Gy. Resolving these equations would then show that the real induction rate for RIF is in fact k′ 1=β·k₁, where k₁ is dose independent and only reflects the time it takes to detect one DSB. The increasing induction rate with doses would then simply reflect β increasing with dose. Our data also show that RIF intensity is larger for higher doses while RIF sizes are similar. This suggests the existence of a well-defined chromatin scaffold for these repair centers, with the presence of multiple DSB requiring more 53BP1 proteins compacted within the same structure. Note, however, that the rigidity of these repair centers is not absolute; this is because we noticed that RIF are both brighter and larger for extremely high doses along HZE tracks.

DNA damage repair centers have been clearly established in Saccharomyces cerevisiae (Lisby et al. (2003) Nat. Cell Biol., 5: 572-577), but they remain hypothetical in mammalian cells, as initially suggested by Savage (Savage (1996) Mutat. Res. 366:81-95; Savage (1996) Mutat. Res., 512: 93-109). However, there are some data suggesting their existence in human cells. For example, there were indications in human blood cells that chromosomal rearrangements observed after exposure to high LET could be explained by localized movement of chromatin containing damaged DNA into local repair centers (Anderson et al. (2002) Proc. Natl. Acad. Sci. USA, 99: 12167-12172). Following up on this work, it was more recently shown that increasing LET of an a particle did not increase the total number of aberrations per track traversal, and instead increased the ratio of complex to total aberrations (Anderson R M, et al. (2007) Radiat. Res. 167: 541-550). Therefore, if DSB clustering occur, as LET goes up (for LET>100 keV/μm), RIF linear frequencies would not change significantly but each RIF would be made of more DSBs, increasing the probability of complex chromosomal rearrangements. In agreement with this theoretical argument, high-resolution imaging of high-LET tracks in combination with Monte Carlo simulation have suggested recently the presence of multiple DSBs within one single RIF (Du et al. (2011) Radiat. Res. 176(6): 706-715). Similarly, a recent theoretical follow-up study taking into account the track structure of high-energy ions and the supercoiled topography of DNA confirmed that multiple DSB can be contained within one single RIF (Ponomarev et al. (2008) Int. J. Radiat. Biol. 84: 916-929). Finally, we previously showed that spatial RIF distribution along high-LET tracks implied relocalization of DSBs rapidly post-IR (Costes et al. (2007) PLoS Comput. Biol. 3: e155), and an independent study reached the same conclusion as 53BP1 RIF pattern along tracks differed significantly from theoretical expectations assuming a simple model of homogenous chromatin distribution (Hauptner A, et al. (2006) Radiat. Prot. Dosimetry, 122: 147-149). The data presented herein bring additional evidence of the existence of repair centers in human (and presumably other mammalian) cells.

RIF Resolution Kinetics Reflect Both Break Complexity and Break Density.

If we were to accept the classic definition that a complex DSB is made by at least three single-strand breaks within 10 base pairs (Nikjoo et al. (1997) Int. J. Radiat. Biol., 71: 467-483), then it is estimated that 20 to 30% of DSBs are complex after exposure to low-LET radiations. In contrast, 70% of the damage induced by the ion used in this work is complex (Nikjoo et al. (2001) Radiat. Res. 156: 577-583). The resolution kinetics constants reported here show large difference of resolution kinetics between these two radiation qualities, with half-lives for RIF resolution as fast as 1.4 h after 0.1 Gy of X-rays and as slow as 10 h after high-LET for an estimated local dose of 26 Gy along Fe ions tracks. In comparison, using PFGE after higher doses of X-ray (>10 Gy), the fast repair half-life associated with simple DSB is approximately 5-30 min and the slow repair half-life is approximately 4-10 h (Wang et al. (2001) Oncogene. 20: 2212-2224; Karlsson et al. (2008) Radiat. Res. 169: 506-512). Therefore, even though RIF resolution does not only reflect DSB repair, but delays due to the clearing of 53BP1 after repair (Kato et al. (2008) Mutat. Res. 639: 108-112; Leatherbarrow et al. (2006) Int. J. Radiat. Biol. 82: 111-118), IR-induced DSB repair kinetics correlate well with RIF disappearance. Classically, the different DSB repair kinetics between different LET has been interpreted as additional delays for repairing complex DSBs. However, our work suggests that using the same LET, local dose effects alone can affect resolution kinetics: There is a 4-fold increase in RIF resolution half-lives between 0.1 and 2 Gy of X-rays (5.7 h at 2 Gy). Therefore, we conclude that slower DSB repair kinetics may not only reflect the presence of complex breaks, but also the presence of multiple DSB within one repair center, leading to a repair machinery having difficulty handling multiple ends of DNA strands in the same location.

High RIF Yield at Low Dose for MCF10A.

Under normal conditions, we detect many more RIF than expected in MCF10A after 0.1 Gy (64 RIF/Gy detected vs 35 RIF/Gy expected), especially in live cell imaging (73 RIF/Gy). Note that this leads to β value less than 1. This effect seems to be cell dependent: similar but more modest yields were observed for live imaging of fibrosarcoma cells HT1080 with 49 RIF/Gy and 40 RIF/Gy following 0.05 and 0.1 Gy, respectively; and 30 RIF/Gy following 0.1 Gy in fixed normal human skin HCA2. In addition, we have not confirmed that the increase of RIF yield at low dose correlates with other surrogate markers of DNA damage such as chromosomal aberrations or micronuclei.

We also show here that ATM inhibition result in 3-fold reduction of a after 0.1 Gy of X-rays with a yield of 25 RIF/Gy, whereas no significant reduction of a is observed after 2 Gy with a yield of 12 RIF/Gy comparable to 15 RIF/Gy under normal conditions. This suggests that the higher RIF yield at low-dose IR is ATM dependent. Because IR can induce heterochromatin decondensation in Drosophila cells (Chiolo et al. (2011) Cell, 144: 732-744) or in mammalian cells (Jakob et al. (2011) Nucleic Acids Res. 39: 6489-6499), one could thus hypothesize that low doses of IR induce a global but subtle chromatin reorganization, which could lead to increase foci that may not necessarily relate to more DNA damage. In agreement with this hypothesis, ATM has been shown to autophosphorylate and consequently phosphorylate H2AX when nuclear volumes are dilated by using hypotonic media (Bakkenist and Kastan (2003) Nature 421: 499-506). Similarly, it has been shown that hypotonic conditions alone are sufficient to induce binding of 53BP1 to chromatin (Baure et al. (2009) Mutagenesis 24: 161-167).

Impact of Results for Regulating Risk of IR on Human Populations.

The current literature has assumed the linear-no-threshold hypothesis (LNT), which implies that any amounts of IR are harmful. LNT is used to set dose limits for radiation occupational workers or the general public. The LNT is based mainly on data from the Japanese atomic bomb survivors and secondarily on arguments involving the dose-response of surrogate endpoints. Gene mutations are thought to be the initiating events of cancer and they can occur via misrejoining of two DNA DSBs or via point mutation. Physical laws lead us to believe DSB frequencies are proportional to dose. Therefore, it is well accepted that point mutations are linear with dose because it requires only one DSB, whereas DSB misrejoinings are dependent to the dose squared (Costes et al. (2001) Radiat. Res., 156: 545-557). In the dose range of radiation cancer epidemiology, the quadratic term is almost always negligible, especially at low dose rates, as the first lesion is probably repaired before the second mutation occurs (Brenner and Sachs (2006) Radiat. Environ. Biophys. 44: 253-256). However, the amount of DSB clustering at 1 Gy suggests a much higher quadratic term for DSB misrejoining than expected. Therefore, extrapolating risk linearly from high dose as done with the LNT could lead to overestimation of cancer risk at low doses.

### Materials and Methods

Cell Culture.

Nonmalignant human mammary epithelial cells (MCF10A, purchased from ATCC) were grown on 8-well Lab-Tek chambered coverglass (Nalge Nunc International) or on 48-spot functionalized glass slides (AmpliGrid, Beckman Coulter GmbH). The cells were grown until they formed a monolayer (approximately 85% confluent) prior to irradiation. See Supporting Information Materials and Methods and FIG. 16 for full details.

Irradiation and ATM Inhibition.

The cells were fixed for immunofluorescence at specific intervals after exposure to X-rays. We typically refer to “low dose” or “high dose” as doses below or equal to 0.1 Gy or larger than 1 Gy, respectively. For high-LET IR, cells were irradiated at the accelerator beam line of the National Aeronautics and Space Administration Space Research Laboratory at Brookhaven National Laboratory. ATM activity was inhibited by incubating cells with 10 μM of ATM specific inhibitor KU55933 (Calbiochem) from 1 h pre-IR until cells were fixed, as previously described (Hickson et al. (2004) Cancer Res. 64: 9152-9159).

Immunostaining and Imaging.

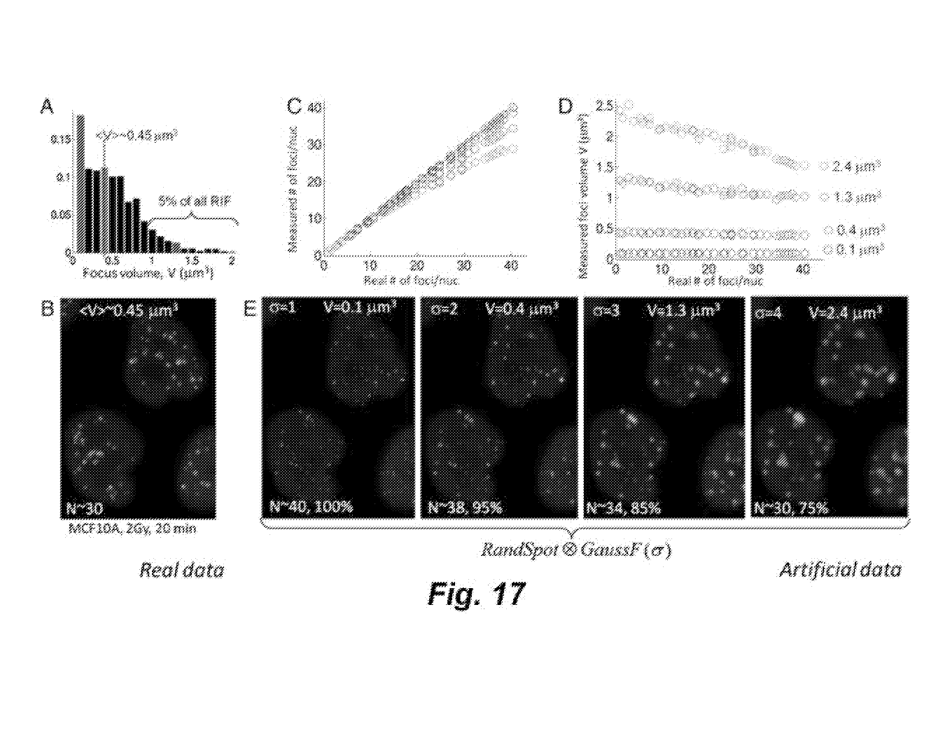

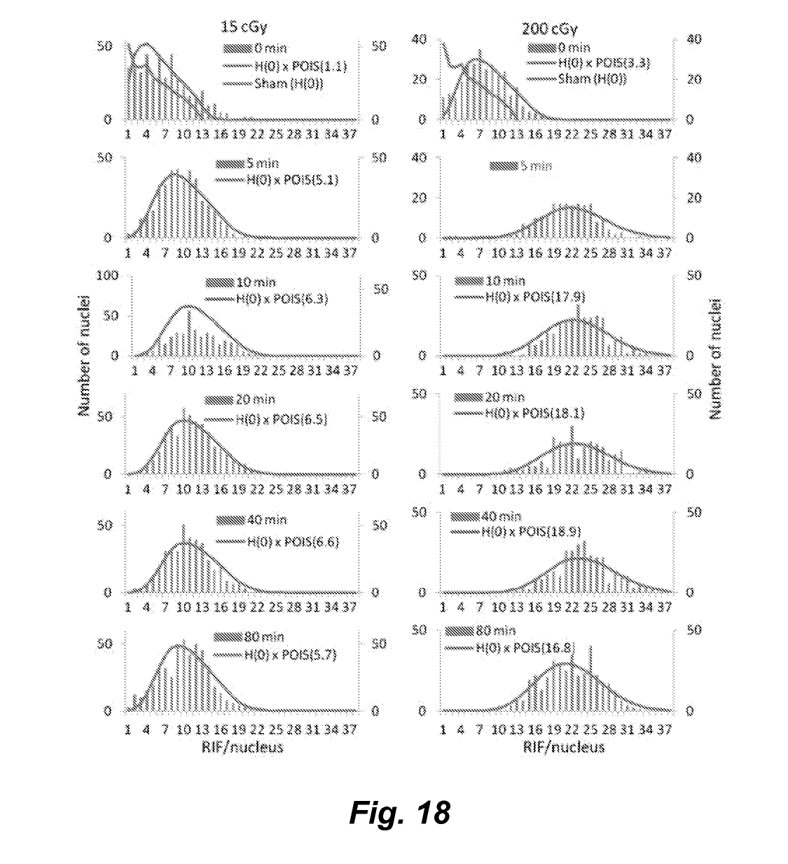

We are only briefly describing these procedures. For complete information, see Supplemental Information Materials and Methods. Immunostaining using anti-53BP1 (rabbit polyclonal, Bethyl Laboratories A300-272A) was performed according to previous staining protocol (Costes et al. (2006) Radiat. Res. 165: 505-515). For image acquisition, both live and fixed MCF10A were imaged using a Zeiss plan-apochromat 40× dry objective (N.A. of 0.95) at a fixed exposure time. Nondeconvolved 3D stacks were acquired and used for image analysis (10 slices of 0.5-μm steps for fixed cells and 3 slices of 1-μm step for live cells). All image manipulations, foci analysis, and statistics were done with Matlab (MathWorks, Inc.) and DIPimage (image processing toolbox for Matlab, Delft University of Technology). In contrast to previous intensity-based methods for RIF identification (Bocker and Iliakis (2006) Radiat. Res. 165: 113-124), we used a pattern recognition approach to detect RIF by applying a wavelet morphological filter to enhance RIF peaks in the image while reducing noise from nonspecific signals (Olivo-Marin (2002) Pattern Recognition 35: 1989-1996). Nuclear space occupied by RIF was identified by applying a constant threshold on the wavelet filtered image, and watershed algorithm was used to separate touching RIF. To test if focus size could affect the accuracy of automatic RIF detection, we applied the software on simulated data where foci sizes and densities had different values (i.e., 1 to 40 foci/nucleus were simulated with four distinct sizes: 0.1, 0.4, 1.3, and 2.4 μm³; FIG. 17). We concluded that foci overlap at the highest foci density (40 foci/nucleus) would be negligible in real data and therefore would not impact RIF counts. Finally, in order to extract the number of “real” RIF from the number of background foci in each scored nucleus, we introduced a background subtraction method that assumes the measured RIF distribution is the result of a convolution between the “real” RIF distribution and the background foci distribution (FIG. 18). For quantification of RIF in live cells, we counted both the cumulative and instantaneous number of RIF manually in 3D time-lapse images. Time interval varied between experiments and was generally set to 10-min interval for the first hour, followed by 30-min interval afterwards. This setting was optimum to minimize phototoxicity and specimen bleaching. Because of the difficulty of software to track individual foci in successive time lapse, analysis had to be done manually in a blind manner on processed images.

Mathematical Model of DSB Detection and RIF Formation.

In order to interpret RIF kinetics in an unbiased manner, we introduce a simple mathematical model describing RIF formation where one DSB is detected at a rate k leading to one RIF, and one RIF is resolved after repair at a rate k₂, assuming both processes are irreversible. This model can be noted as follows:

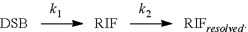

Let C₀ and C₁ be the average number of DSB and RIF per nucleus at time t, respectively. This kinetic model translates then into the following set of differential equations:

$$\{ \begin{matrix}\frac{d {C}_{0}}{d t} = - {k}_{1} {C}_{0} \\ \frac{d {C}_{1}}{d t} = {k}_{1} {C}_{0} - {k}_{2} {C}_{1}\end{matrix} {⇒}^{{C}_{1} ( 0 ) = 0} \{ \begin{matrix}{C}_{0} ( t ) = α D . {e}^{- {k}_{1} t} \\ {C}_{1} ( t ) = \frac{α {Dk}_{1}}{{k}_{2} - {k}_{1}} ( {e}^{- {k}_{1} t} - {e}^{- {k}_{2} t} )\end{matrix} \qquad [ 1 ]$$

where α is the number of naked DSB/Gy before formation of RIF and D is the dose delivered to the cell. Alpha (α) should be constant for all doses. Further details are provided in the supporting information materials and methods regarding the way Eq. 1 is fitted. Note that one could modify the kinetic model presented here to separate rapid repair of simple lesions and slow repair of complex lesions as it has been previously suggested from PFGE DSB kinetics (Wang et al. (2001) Oncogene. 20: 2212-2224; Karlsson et al. (2008) Radiat. Res. 169: 506-512). This would, however, lead to an additional kinetic constant, that would result in multiple solutions for the same fit. We therefore opted for a mathematical model that can be resolved with less ambiguity, using only one rate for induction and one rate for resolution.

C₁(t) in Eq. 1 can be used to fit the number of RIF at a given time (static measure). However, one can also measure using time-lapse imaging the total number of RIF that have been produced since t=0 (cumulated measure). This can be described mathematically as

C_{c}(t)=αD(1−e^{-k}^{₁}^{t}).

Eq. 2 is derived simply by setting k₂=0 and using the same formalism as in Eq. 1. Note that the corresponding half-life for k₁ and k₂ (i.e., t_{1/2}k=ln (2)/k) are reported in the text. t_{1/2}k₁ represents the time it takes for half of all DSBs to be detected as RIF. t_{1/2k2} represents the time it takes for half of the total number of RIF to be resolved.

### Supporting Information Materials and Methods

Cell Culture.

Adherent growing human foreskin diploid fibroblasts (HCA2) were cultivated in minimum essential medium (MEM) alpha (Invitrogen Inc.), supplemented with 10% fetal bovine serum. Human mammary epithelial cells, MCF10A obtained from ATCC, were grown in MEMB media supplemented with bovine pituitary hormone (13 mg/mL), hydrocortisone (0.5 mg/mL), hEGF (10 μg/mL), insulin (5 mg/mL), and cholera toxin (100 ng/mL) (Invitrogen Inc.). Both cell lines were grown at 37° C., with 95% humidity and 5% CO₂. For experiments, both cell lines were seeded either in Permanox plastic 8-well Lab-Tek chamber slides (Nalge Nunc International Corporation) or on 48 hydrophilic spots of functionalized glass-slides (AmpliGrid, Beckman Coulter GmbH). The cells were grown to a confluent layer prior to irradiation. HT1080 and human bronchial epithelial cells (HBEC) were grown and maintained as previously described (Costes et al. (2006) Radiat. Res. 165: 505-515). For live cell imaging, HT1080 and HBEC were stably transfected with 53BP1-GFP (1), whereas MCF10A were transiently transfected with H1.5-DsRed2 and 53BP1-GFP using lipofectamine LTX (Invitrogen). H1.5-DsRed2 for chromatin labeling was generously given by Michael Hendzel from the University of Alberta, Canada. DNA damage labeling was done with 53BP1-GFP construct, generously given by Thanos Halazonetis from the University of Geneva, Switzerland.

Irradiation.

Identical dose- and time-response experiments were conducted with cells exposed to X-rays (160 or 320 kV) to optimize the immunostaining of radiation-induced foci. For the optimization experiments, cells were irradiated with 100 cGy of X-ray and fixed after 30-min repair time to get a maximum radiation-induced foci (RIF) induction as previously shown (Costes et al. (2007) PLoS Comput. Biol. 3: e155). For the matrix experiments with different doses and time responses, cells grown on one functionalized glass slide were irradiated with two doses. Therefore, one part of the modified glass slide was shielded with lead. Furthermore, the sample was placed on top of lead to minimize backscattering. Cells in each well were fixed at different time and dose points (0, 1, 5, 10, 20, 40, 80 min post—IR/0, 5, 10, 15, 40, 50, 100, 200, and 400 cGy) on a warm block and returned to the 37° C. incubator. Dose rates were modified as little as possible for each dose as long as the exposure time was less than 1 min to get accurate early time points, and was more than 10 s for accurate determination of the dose. This led to three different dose rates: 450 cGy/min for 200 and 400 cGy; 150 cGy/min for 100, 50, and 40; 30 cGy/min for 5, 10, and 15 cGy. For high-LET radiation, cells were irradiated at the accelerator beam line of the National Aeronautics and Space Administration Space Research Laboratory at Brookhaven National Laboratory, with either 1 GeV/atomic mass unit Fe ions or 1 GeV/atomic mass unit 0 ions (LET=150 keV/μm and 14 keV/μm, respectively). A dose of 1 Gy was delivered at a dose rate of 100 cGy/min.

Immunostaining.

Two different culture platforms were evaluated (i.e., 48-microwell ampligrid vs. 8-well chamber slides). Immunostaining was optimized using cells exposed to 100 cGy of X-rays and fixed 30 min after irradiation with 2% paraformaldehyde in PBS for 15 min at room temperature followed by permeabilization with 100% ice-cold methanol for 15 min at −20° C. Subsequently, blocking, primary antibody incubation, and secondary antibody incubation were optimized through titration experiments. The rest of the staining was performed according to the conventional staining protocol (Costes et al. (2007) PLoS Comput. Biol. 3: e155) but with BSA used for blocking instead of casein supernatant. When cells were grown on Ampli-Grids, optimization was performed reducing the immunostaining time to less than 1 h. By using 5 μL of reagent for each incubation step in the microwells, we could increase antibody concentration with no significant impact on cost. Briefly, titration times for the optimization were 1, 2, 4, 8, 16, 32, or 64 min as well as additional 128 min for the primary antibody. The primary antibodies were either a rabbit polyclonal anti 53BP1 antibody (stock at 1 mg/mL, Bethyl Laboratories) or a mouse monoclonal to phosphohistone H2AX antibody (stock at 1 mg/mL, clone JBW301; Upstate Cell Signalling Solutions Inc.). The corresponding secondary antibodies were either FITC labeled antirabbit IgGs or, FITC or T-Red labeled antimouse IgGs (Molecular Probes Invitrogen). After three washing steps with PBS at room temperature, cells were either blocked with 0.1% BSA for 1 h for the antibody titers or the blocking titer was performed with 0.1%, 0.2%, and 1% BSA at room temperature. The samples of the blocking titer were incubated with the primary antibody for 2 h and then, after extensive washing with PBS, incubated with the secondary antibody for 1 h. The other samples were either incubated with the primary antibody for 2 h and, subsequently, used for the secondary antibody titer, or the primary titer with the dilutions 1:10, 1:100, 1:200 was performed at room temperature. The primary titer samples were washed extensively with PBS after the titration and then incubated with the secondary antibody for 1 h. The secondary antibody titer samples were also washed with PBS before the secondary antibody titration was performed. Dilutions used for the secondary antibody incubation optimization were 1:10, 1:100, 1:200. After a further washing step with PBS, the samples were counterstained with DAPI and then analyzed with regard to foci intensity.

By plotting the relative foci intensities against time, saturation of the relative foci intensities was observed after a short time for all concentrations and dilutions. Saturation was reached for the blocking titration between 8-16 min in dependence on the BSA concentration. For the primary antibody, the saturation was always reached after 16 min independent from the antibody concentration. The secondary antibody plot showed more variations in the saturation time points in dependence on the antibody concentration. Saturation was obtained after 32 min for the 1:200 antibody dilutions, after 16 min for the 1:100 dilution, and after 8 min for the 1:10 dilution. FIG. 16, panel A shows the progression of the curves for the 1:100 dilution for both antibodies and the curve for the 1% BSA solution. The curve progressions as well as the intensity of the microscopic images led to the conclusion that longer incubation does not improve the quality of the images. Indeed, longer blocking results in lower foci intensities (FIG. 16, panel B). For both antibodies, the saturation in the foci intensity can also be seen in the microscopic images (FIG. 16, panels C and D). The saturation of the titration curves observed as well as the quality of the images led to the decision to reduce the incubation time for the three staining steps to 15 min for the three reagents and to use a 0.2% concentration of BSA and 1:100 dilutions for both antibodies in the matrix experiments. Corresponding images for these incubation times and dilution are shown in FIG. 16, panel E, clearly showing the improvement in image quality compared to other conditions (FIG. 16, panels B-D).

Image Acquisition.

Cells were viewed and imaged using a Zeiss Axiovert 200M automated microscope with Ludl position-encoded scanning stage (Carl Zeiss). Images were acquired using a Zeiss plan-apochromat 40× dry objective (N.A. of 0.95) and a very sensitive scientific-grade EM-CCD camera (Hamamatsu C9100-02, 1,000 by 1,000 pixels, 8×8 μm2 pixels). The image pixel size was measured to be 0.2 μm but based on the NA of the objective, the actual resolution of the image in the FITC channel is approximately 0.5×0.488/NA=0.26 μm. All images were captured with the same exposure time so that intensities were within the 16-bit linear range and could be compared between specimens. For 3D dataset, a CSU-10 spinning disk confocal scanner was used to acquire optical slices of 0.5-μm thickness, and illumination was provided by four solid-state lasers at 405, 491, 561, and 638 nm under AOTF control (Acousto-Optic Tunable Filters). For 2D dataset, simple conventional image was taken with the same optics but without spinning disk. Finally, a multiband dichroic and single-band emission filters in a filter wheel selected the fluorescent light captured by the camera, removing any type of bleed through between channels. For X-ray experiments on live HT1080, time-lapse imaging was carried out as previously described (Costes et al. (2006) Radiat. Res. 165: 505-515), using an LSM 510 Meta laser scanning confocal microscope (Carl Zeiss) with a 63×1.4 NA Plan-Apochromat oil immersion objective.

Cell Cycle Considerations.

We noted that MCF10A are not fully arrested at confluence, and thus we corrected for high foci count from cells in G2 or S phase as previously described (Costes et al. (2007) PLoS Comput. Biol. 3: e155). Briefly, foci counts were scaled to represent the number of foci for the same size nucleus, using the G1 nuclear volume as the reference nuclear volume. DAPI content and EdU pulsing (Click-iT®, Invitrogen) were used to estimate proportions of cells in each phase. Note that cells in late G2 are problematic as 53BP1 signals becomes weaker with a signal fully cytoplasmic during mitosis, leading to complete loss of foci until reentry in G1. However, this effect should have very little impact on the analysis because only 5% of cells were in G2 and less than 1% in mitosis. We also measured 9% of the cells being in S phase, which could lead to higher foci background due to stalled replication forks. However, working with 53BP1 alleviated this problem, as background issues have been reported primarily with γH2AX not 53BP1 (Id.).

Image Analysis of Live Cells.

Processing of 3D time lapse was done by first applying a maximum intensity projection (MIP) on all Z stack to allow visualization of all foci within one single plane. This first step resulted in the generation of 2D time lapse, which could then be realigned between time points on a per nucleus basis (translation and rotation), to help distinguishing foci movement from foci formation or resolution. Various doses of X-rays were considered (0.05, 0.1, and 1 Gy) for a kinetic covering 5 min to 20 h post-IR, depending on the cells used. 3D time lapses were acquired and averaged over 20 and 40 cells for each dose. RIF size for live cell imaging was obtained by computing the full width at half maximum determined by a 1D intensity profile crossing the center of the RIF. The cross-section was done manually, and the reported size only reflected the average diameter of the RIF.

Impact of Foci Size and Foci Density on Foci Detection.

Nuclear space occupied by RIF was identified by applying a constant threshold on the wavelet filtered image, and watershed algorithm was used to separate touching RIF. To test if focus size could affect the accuracy of automatic RIF detection, we applied the software on simulated data where foci sizes and densities had different values (i.e., 1 to 40 foci/nucleus were simulated with four distinct sizes: 0.1, 0.4, 1.3, and 2.4 μm³, FIG. 17). We concluded that foci overlap at the highest foci density (40 foci/nucleus) will be negligible in real data and therefore will not impact RIF counts. When foci were all as large as 1.3 or 2.4 μm³, we started computing number of foci/nucleus lower than simulated (i.e., 10% and 25% lower than expected, respectively, when simulating 40 foci/nucleus). It is interesting to note in that situation the algorithm reported lower sizes than simulated as well. This reflects the ability of the algorithm to separate touching foci, minimizing the impact of foci overlap. Because RIF sizes are on average much lower (i.e., 95% of RIF sizes in a real specimen exposed to 1 Gy are below 1 μm³; FIG. 17, panel A), and the minimum detectable focus size is approximately 0.1 μm³, simulations suggest that foci overlap at the highest foci density (40 foci/nucleus) will be negligible in real data and therefore will not impact RIF counts. For quantification of RIF in live cells, we counted both the cumulative and instantaneous number of RIF manually in 3D time-lapse images. Time interval varied between experiments and was generally set to 10 min interval for the first hour, followed by 30 min interval afterward. This setting was optimum to minimize phototoxicity and specimen bleaching. Because of the difficulty of software to track individual foci in successive time lapse, analysis was done manually in a blind manner on processed images.

Background Foci Correction.

The human cells we used have significant amount of background foci. In this work, we introduced a method to correct for their presence in irradiated specimen. Briefly, we know that DNA damages are random events taking place in a specified unit of space (the nucleus) with an average frequency Φ (RIF/nucleus). Therefore, the probability of having N hits in a given cell is defined by the Poisson distribution Pois(N/Φ). If we were to measure the number of cells with N RIF after exposure of a dose D, this would lead to the distribution H(N,D)=H(N,0) [symbol] Pois(N,Φ), where H(N,0) is the distribution of background foci without radiation. In other words, the measured distribution of RIF/nucleus in a specimen should be a Poisson distribution whose means is the average number of RIF/nucleus convolved with the distribution of background foci present before exposure to ionizing radiation. For each measured distribution H(N,D), we searched the value of Φ that yielded the best fit by incremental changes on Φ.

If the Poisson assumption is right, such method should lead to more accurate values for RIF estimation (i.e., fitting with a mathematical function is less sensitive to noise than computing the average). High R squared values between the fits and the measured distributions were indeed observed (average R²˜0.92; FIG. 18), validating the assumption that “real” RIF are distributed randomly among nuclei, much like double strand break (DSB). This background correction worked well down to 0.15 Gy (average R2˜0.93). However, 0.05 Gy exposures led to distributions that could not be fitted with high statistical significance, a problem that might be overcome with much larger sample sizes. We are, therefore, only reporting RIF frequencies for doses ≧0.15 Gy. One should also note that correcting the measured number of RIF by only subtracting the mean number of background foci could not have been fitted well by a Poisson distribution due to the non-Poisson contribution of background foci (downward sloping line top graphs, FIG. 18). It is known that background foci changes with each cell cycle and the nonnormal distribution probably reflects the various cycle distribution. Therefore, such traditional method would not have permitted us to conclude on the random distribution of RIF.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A system for determining the sensitivity of cells to ionizing radiation or to non-ionizing radiation, said system comprising:
a microfluidics device comprising a plurality of microfluidic cavities each configured to contain cells;
a source of ionizing radiation or non-ionizing radiation configured to deliver said radiation to cells in said microfluidic cavities; and
an imaging system configured to detect radiation-induced foci in said cells when they are disposed in said microfluidic cavities.

2. The system of claim 1, wherein said source of radiation is a source of ionizing radiation.

3. The system of claim 2, wherein aid source of radiation is a radionuclide or an x-ray source.

4. The system of claim 2, wherein said source of radiation is an x-ray source.

5. The system of claim 2, wherein said source of ionizing radiation is a mini X-ray tube.

6. The system of claim 1, wherein said source of radiation is a source of non-ionizing radiation.

7. The system of claim 6, wherein said source of non-ionizing radiation is a UV source.

8. The system according to any one of claims 1-7, wherein said microfluidic device comprises at least eight microcavity cells for each sensitivity determination that is to be performed.

9. The system of claim 8, wherein said microfluidic device is configured to provide a plurality of sensitivity determinations.

10. The system of claim 9, wherein said microfluidic device is configured to provide at least four different sensitivity determinations.

11. The system according to any one of claims 8-10, wherein the at least eight microcavity cells for each sensitivity determination are disposed along a line on said microfluidic device.

12. The system according to any one of claims 1-11, wherein said microfluidic device is operably coupled to or further comprises a cell separator.

13. The system of claim 12, wherein said cell separator is configured to separate lymphocytes from a blood or blood fraction sample and deliver said lymphocytes into the microfluidic cavities.

14. The system of claim 13, wherein said separator lyses erythrocytes and isolates leukocytes.

15. The system according to any one of claims 12-14, wherein channels or chambers in said cell separator are coupled to said microcavities by microchannels and configured to deliver said lymphocytes from said separator into said microcavities.

16. The system according to any one of claims 1-15, wherein said microfluidics device comprises a fabricated block within which are formed, embedded or molded, one or more fluid-tight channels.

17. The system of claim 16, wherein the block material from which the device is fabricated is selected from the group consisting of polydimethylsiloxane (PDMS), polyolefin plastomer (POP), perfluoropolyethylene (PFPE), polyurethane, polyimides, and cross-linked NOVOLAC® (phenol formaldehyde polymer) resins, glass (including, but not limited to, borosilicate glass, SF11, and SF12), quartz, cyclic olefin copolymers (COC), cyclic olefin polymers (COP), acrylate polymers, polystyrene and polycarbonate.

18. The system according to any one of claims 1-17, further comprising a pump or pressure system to move cells and/or reagents through or into said microchannels and/or said microcavities.

19. The system according to any one of claims 1-18, wherein said imaging system comprises a digital camera.

20. The system according to any one of claims 1-19, wherein said imaging system comprises a microscope objective.

21. The system according to any one of claims 1-20, wherein said microfluidic device is configured on a movable stage to move said device with respect said microscope objective so that different microcavities can be imaged by the same objective.

22. The system according to any one of claims 1-21, wherein said microscope objective can be moved with respect to said microfluidic device to permit alignment of said objective with different microcavities.

23. The system according to any one of claims 1-22, further comprising one or more detection reagents to label radiation induced foci in cells.

24. The system of claim 23, wherein said detection reagents comprise labeled antibodies that bind to radiation induced foci.

25. The system of claim 24, wherein said antibodies are selected from the group consisting of anti-P53 binding protein 1, anti-γH2AX, anti-Rad51, anti-MRE11, anti-XRCC1, anti-Rad50, anti-BRCA1, anti-ATM, anti-ATR, and anti-DNApkcs.

26. The system according to any one of claims 1-25, wherein said system is operably connected to a computer.

27. The system of claim 26, wherein said computer is configured to quantify radiation-induced foci in images acquired by said imaging system.

28. The system according to any one of claims 26-27, wherein said computer is configured to determine a repair kinetic for radiation induced foci (RIF) using a model where one double strand break (DSB) is detected at a rate k₁ leading to the formation of one RIF and one RIF is resolved after repair at rate k₂ assuming that both processes are irreversible where the model can be expressed by the equations:
$$\{ \begin{matrix}\frac{d {C}_{0}}{d t} = - {k}_{1} {C}_{0} \\ \frac{d {C}_{1}}{d t} = {k}_{1} {C}_{0} - {k}_{2} {C}_{1}\end{matrix} {⇒}^{{C}_{1} ( 0 ) = 0} \{ \begin{matrix}{C}_{0} ( t ) = α D . {e}^{- {k}_{1} t} \\ {C}_{1} ( t ) = \frac{α {Dk}_{1}}{{k}_{2} - {k}_{1}} ( {e}^{- {k}_{1} t} - {e}^{- {k}_{2} t} )\end{matrix}$$
where C₀ and C₁ are the average number of DSB and RIF per nucleus at time t, respectively, α is the number of naked DSB/Gy before formation of RIF and D is the radiation dose delivered to the cell.

29. The system according to any one of claims 26-28, wherein said computer is further configured to perform one or more actions selected from the group consisting of operating said image analysis system to capture an image, adjusting the field location and/or focus of said microscope objective, determining the location of cells and/or cellular nuclei within an acquired image, controlling the passage of cells and/or reagents into and/or through said microfluidic device.

30. A method of determining the sensitivity of a subject to ionizing radiation and/or to non-ionizing radiation and/or risk of adverse consequences of said radiation to said a subject, said method comprising:
contacting a biological sample comprising cells from said subject with ionizing or non-ionizing radiation;
detecting and quantifying radiation induced foci in said cells at least two different time points; and
determining a repair kinetic for said radiation induced foci that is a measure of the rate of disappearance of said foci, wherein a longer repair kinetic indicates a greater sensitivity of said subject to radiation.

31. The method of claim 30, wherein said contacting comprises contacting said sample to ionizing radiation.

32. The method according to any one of claims 30-31, wherein said ionizing radiation is produced by a radionuclide or by an x-ray source.

33. The method of claim 30, wherein said contacting comprises contacting said sample to non-ionizing radiation.

34. The method according to claims 30 and 33, wherein said non-ionizing radiation source is a UV source.

35. The method according of any one of claims 30-34, wherein high dose radiation is used and said repair kinetic provides a measure of acute response to radiation.

36. The method according to any one of claims 30-34, wherein high dose and low dose radiation is used and said repair kinetic provides a measure of cancer risk.

37. The method according to any one of claims 30-36, wherein said contacting, detecting, and determining is performed using a system according to any one of claims 1-29.

38. The method according to any one of claims 30-37, wherein said repair kinetic for radiation induced foci (RIF) is determined using a model where one double strand break (DSB) is detected at a rate k₁ leading to the formation of one RIF and one RIF is resolved after repair at rate k₂ assuming that both processes are irreversible where the model can be expressed by the equations:
$$\{ \begin{matrix}\frac{d {C}_{0}}{d t} = - {k}_{1} {C}_{0} \\ \frac{d {C}_{1}}{d t} = {k}_{1} {C}_{0} - {k}_{2} {C}_{1}\end{matrix} {⇒}^{{C}_{1} ( 0 ) = 0} \{ \begin{matrix}{C}_{0} ( t ) = α D . {e}^{- {k}_{1} t} \\ {C}_{1} ( t ) = \frac{α {Dk}_{1}}{{k}_{2} - {k}_{1}} ( {e}^{- {k}_{1} t} - {e}^{- {k}_{2} t} )\end{matrix}$$
where C₀ and C₁ are the average number of DSB and RIF per nucleus at time t, respectively, α is the number of naked DSB/Gy before formation of RIF and D is the radiation dose delivered to the cell.

39. The method according to any one of claims 30-38, wherein said repair kinetic is evaluated with respect to the same kinetic determined for said subject at an earlier time and an increase in said kinetic indicates increasing radiation susceptibility of said subject over time.

40. The method according to any one of claims 30-38, wherein said repair kinetic is evaluated with respect to the same kinetic determined for a population or subpopulation and a repair kinetic longer than the average or median repair kinetic for said population or subpopulation indicates that said subject has elevated radiation sensitivity and a repair kinetic shorter than the average or median repair kinetic for said population or subpopulation indicates that said subject has reduced radiation sensitivity.

41. The method of claim 38, wherein risk is evaluated by α, wherein alpha reflects DSB clustering and the lower alpha the higher the risk.

42. The method according to any one of claims 1-41, wherein sensitivity or risk is identified at two different radiation doses, wherein the different sensitivity or risk determined at each dose provides a measure of sensitivity or risk for low dose exposures and for high dose exposures.

43. The method according to any one of claims 30-42, wherein said repair kinetic is normalized to an average or to a median value for a population or subpopulation.

44. The method of claim 43, wherein said repair kinetic is normalized to a subpopulation and said subpopulation comprises members grouped/selected by one or more factors selected from the group consisting of ethnicity, age, gender, occupation, and disease state.

45. The method according to any one of claims 30-44, wherein said cells comprise cells selected from the group consisting of erythrocytes, lymphocytes, primary cells from biopsies.

46. The method according to any one of claims 30-45, wherein said cells are cells from a human.

47. The method of claim 46, wherein said cells are from a human that is to be subjected to radiotherapy and/or medical imaging.

48. The method of claim 46, wherein said cells are from a human that works in a region subject to radiation risk.

49. The method according to any one of claims 30-45, wherein said cells are cells from a non-human mammal.

50. The method of claim 49, and said non-human mammal is a mammal selected from the group consisting of a non-human primate, a canine, a feline, a bovine, an equine, a porcine, and a lagomorph.

51. The method according to any one of claims 30-50, wherein said repair kinetic and/or a diagnosis/prognosis based, at least in part, on said repair kinetic is recorded in a patient medical record.

52. The method of claim 51, wherein said patient medical record is maintained by a laboratory, physician's office, a hospital, a health maintenance organization, an insurance company, or a personal medical record website.

53. The method according to any one of claims 30-50, wherein said repair kinetic and/or a diagnosis/prognosis based, at least in part, on said repair kinetic is recorded on or in a medic alert article selected from a card, worn article, or radiofrequency identification (RFID) tag.

54. The method according to any one of claims 30-50, wherein said repair kinetic and/or a diagnosis/prognosis based, at least in part, on said repair kinetic is recorded on a non-transient computer readable medium.

55. The method according to any one of claims 30-54, wherein when said measure indicates a heightened radiation sensitivity of said subject, as compared to a reference population, adjusting life style and dietary habits as preventive measures.

56. A method of determining the sensitivity of a subject to ionizing radiation and/or to non-ionizing radiation and/or risk of adverse consequences of said radiation to said a subject, said method comprising:
providing a biological sample from said subject comprising cells; and
detecting and quantifying baseline foci in said cells to provide a foci number;
where an increase in foci number as compared to a reference foci number determined for said subject at a previous time or for a population indicates elevated sensitivity of a subject to ionizing radiation and/or to non-ionizing radiation and/or risk of adverse consequences of said radiation to said subject and a decrease in foci number as compared to a reference foci number determined for said subject at a previous time or for a population indicates decreased sensitivity of said subject to ionizing radiation and/or to non-ionizing radiation and/or risk of adverse consequences of said radiation to said subject.

57. The of claim 56, wherein said foci number is evaluated with respect to the same foci number determined for said subject at an earlier time and an increase in said foci number indicates increasing radiation susceptibility of said subject over time.

58. The of claim 56, wherein said foci number is evaluated with respect to the same foci number determined for a population or subpopulation and a foci number larger than the average or median foci number for said population or subpopulation indicates that said subject has elevated radiation sensitivity and a foci number lower than the average or median foci number for said population or subpopulation indicates that said subject has reduced radiation sensitivity.

59. The method according to any one of claims 56-58, wherein said foci number is normalized to an average or to a median value for a population or subpopulation.

60. The method of claim 59, wherein said foci number is normalized to a subpopulation and said subpopulation comprises members grouped/selected by one or more factors selected from the group consisting of ethnicity, age, gender, occupation, and disease state.

61. The method according to any one of claims 56-60, wherein said sample comprises whole blood.

62. The method according to any one of claims 56-61, wherein said sample comprises cells selected from the group consisting of erythrocytes, lymphocytes, primary cells from biopsies.

63. The method according to any one of claims 56-62, wherein said cells are cells from a human.

64. The method of claim 63, wherein said cells are from a human that is to be subjected to radiotherapy and/or medical imaging.

65. The method of claim 63, wherein said cells are from a human that works in a region subject to radiation risk.

66. The method according to any one of claims 56-62, wherein said cells are cells from a non-human mammal.

67. The method of claim 66, wherein said non-human mammal is a mammal selected from the group consisting of a non-human primate, a canine, a feline, a bovine, an equine, a porcine, and a lagomorph.

68. The method according to any one of claims 56-67, wherein said foci number and/or a diagnosis/prognosis based, at least in part, on said foci number is recorded in a patient medical record.

69. The method of claim 68, wherein said patient medical record is maintained by a laboratory, physician's office, a hospital, a health maintenance organization, an insurance company, or a personal medical record website.

70. The method according to any one of claims 56-67, wherein said foci number and/or a diagnosis/prognosis based, at least in part, on said foci number is recorded on or in a medic alert article selected from a card, worn article, or radiofrequency identification (RFID) tag.

71. The method according to any one of claims 56-67, wherein said foci number and/or a diagnosis/prognosis based, at least in part, on said foci number is recorded on a non-transient computer readable medium.

72. The method according to any one of claims 56-71, wherein when said measure indicates a heightened radiation sensitivity of said subject, as compared to a reference population, adjusting life style and dietary habits as preventive measures.

73. The method according to any one of claims 56-72, wherein said detecting and quantifying is performed using a system comprising:
a microfluidics device comprising one or a plurality of microfluidic cavities each configured to contain cells; and
an imaging system configured to detect radiation-induced foci in said cells when they are disposed in said one or plurality of microfluidic cavities.

74. The method of claim 73, wherein said microfluidic device comprises at least one, or at least two, or at least four, or at least eight microcavity cells for each sensitivity determination that is to be performed.

75. The method according to any one of claims 73-74, wherein said microfluidic device is operably coupled to or further comprises a cell separator.

76. The method of claim 75, wherein said cell separator is configured to separate lymphocytes from a blood or blood fraction sample and deliver said lymphocytes into the microfluidic cavities.

77. The method according to any one of claims 75-76, wherein channels or chambers in said cell separator are coupled to said microcavities by microchannels and configured to deliver said lymphocytes from said separator into said microcavities.

78. The method according to any one of claims 73-77, wherein said device lyses erythrocytes and isolates leukocytes.

79. The method according to any one of claims 73-78, wherein said microfluidics device comprises a fabricated block within which are formed, embedded or molded, one or more fluid-tight channels.

80. The method of claim 79, wherein the block material from which the device is fabricated is selected from the group consisting of polydimethylsiloxane (PDMS), polyolefin plastomer (POP), perfluoropolyethylene (PFPE), polyurethane, polyimides, and cross-linked NOVOLAC® (phenol formaldehyde polymer) resins, glass (including, but not limited to, borosilicate glass, SF11, and SF12), quartz, cyclic olefin copolymers (COC), cyclic olefin polymers (COP), acrylate polymers, polystyrene and polycarbonate.

81. The method according to any one of claims 73-80, wherein device and/or system comprises a pump or pressure system to move cells and/or reagents through or into said microchannels and/or said microcavities.

82. The method according to any one of claims 73-81, wherein said imaging system comprises a digital camera or camera chip.

83. The method according to any one of claims 73-82, wherein said imaging system comprises a microscope objective.

84. The method according to any one of claims 73-83, wherein said device comprises one or more detection reagents to label radiation induced foci in cells.

85. The method of claim 84, wherein said detection reagents comprise labeled antibodies that bind to radiation induced foci.

86. The method of claim 85, wherein said antibodies are selected from the group consisting of anti-P53 binding protein 1, anti-γH2AX, anti-Rad51, anti-MRE11, anti-XRCC1, anti-Rad50, anti-BRCA1, anti-ATM, anti-ATR, and anti-DNApkcs.

87. The method according to any one of claims 73-86, wherein said system is operably connected to a computer.

88. The method of claim 87, wherein said computer is configured to quantify foci in images acquired by said imaging system.

89. The method according to any one of claims 87-88, wherein said computer is configured to perform one or more actions selected from the group consisting of operating said image analysis system to capture an image, adjusting the field location and/or focus of said microscope objective, determining the location of cells and/or cellular nuclei within an acquired image, controlling the passage of cells and/or reagents into and/or through said microfluidic device.

90. A method of administering radiation therapy to a subject and/or imaging said subject, said method comprising:
receiving a measure of sensitivity to radiation based on a measurement of a sample from said subject according to the method of any one of claims 30-54 or a measure of sensitivity to radiation based on a measurement of a sample from said subject according to the method of any one of claims 56-89; and
where, when said measure indicates a heightened radiation sensitivity of said subject, as compared to a reference population, adjusting the mode of administration of said radiotherapy reduce off-target radiation exposure, and/or to increase recovery times between periods of radiation administration; and/or
where, when said measure indicates a heightened radiation sensitivity of said subject, as compared to a reference population, adjusting the imaging modality to reduce exposure to ionizing radiation.

91. The method of claim 90, wherein said method comprises receiving a measure of sensitivity to radiation based on a measurement of a sample from said subject according to the method of any one of claims 30-54.

92. The method of claim 90, wherein said method comprises receiving a measure of sensitivity to radiation based on a measurement of a sample from said subject according to the method of any one of claims 56-89.

93. The method according to any one of claims 90-92, wherein said method comprises a method of administering radiation therapy to a subject and, when said measure indicates a heightened radiation sensitivity of said subject, as compared to a reference population, the mode of administration of said radiotherapy is adjusted to reduce off-target radiation exposure, and/or to increase recovery times between periods of radiation administration.

94. The method of claim 93, wherein the radiation therapy comprises application of external radiation and said administration is adjusted by increasing the number of exposure directions to improve skin sparing.

95. The method of claim 93, wherein the radiation therapy comprises application of internal radiation and said administration is adjusted by utilizing radioisotope that have a shorter half-life and/or that are lower energy.

96. The method according to any one of claims 93-95, wherein said administration is adjusted by increasing recovery times between rounds of administration.

97. The method according to any one of claims 90-92, wherein said method comprises a method of medical imaging in said subject and, when said measure indicates a heightened radiation sensitivity of said subject, as compared to a reference population, the imaging modality is adjusted to reduce exposure to ionizing radiation.

98. The method of claim 97, wherein said imaging modality is adjusted by utilizing NMR or ultrasound.

99. The method according to any one of claims 90-98, wherein said subject is a human.

100. The method according to any one of claims 90-98, wherein said subject is a non-human mammal.

101. A method of evaluating cancer risk in a subject, said method comprising:
receiving a measure of sensitivity to radiation based on a measurement of a sample from said subject according to the method of any one of claims 30-54 or a measure of sensitivity to radiation based on a measurement of a sample from said subject according to the method of any one of claims 56-89; and
where, when said measure indicates a heightened radiation sensitivity of said subject, as compared to a reference population, said subject is identified as at elevated risk for cancer.

102. The method of claim 101, wherein said method comprises receiving a measure of sensitivity to radiation based on a measurement of a sample from said subject according to the method of any one of claims 30-54.

103. The method of claim 101, wherein said method comprises receiving a measure of sensitivity to radiation based on a measurement of a sample from said subject according to the method of any one of claims 56-89

104. The method according to any one of claims 101-103, wherein when said measure indicates a heightened cancer risk of said subject, as compared to a reference population, adjusting life style and dietary habits as preventive measures.

105. The method according to any one of claims 101-103, wherein said measure of sensitivity to radiation, or a cancer risk based, at least in part, on a measure of sensitivity to radiation, is recorded in a patient medical record.

106. The method of claim 105, wherein said patient medical record is maintained by a laboratory, physician's office, a hospital, a health maintenance organization, an insurance company, or a personal medical record website.

107. The method according to any one of claims 101-103, wherein said measure of sensitivity to radiation, or a cancer risk based, at least in part, on measure of sensitivity to radiation, is recorded on or in a medic alert article selected from a card, worn article, or radiofrequency identification (RFID) tag.

108. The method according to any one of claims 101-103, wherein said measure of sensitivity to radiation, or a cancer risk based, at least in part, on measure of sensitivity to radiation, is recorded on a non-transient computer readable medium.
